(19) United States
(12) Patent Application Publication
(10) Pub. No.: US 2026/0338815 A1
(43) Pub. Date: Oct. 29, 2026

(54) AUTOMATIC DISPENSER FOR GAS MEASURING DEVICES, AND METHOD FOR DISPENSING AND METHOD FOR RETURNING A GAS MEASURING DEVICE

(71) Applicant: Dräger Safety AG & Co. KGaA, Lübeck (DE)

(72) Inventors: Thielo Hammer, Lübeck (DE); Marc Brickwedde, Lübeck (DE)

(21) Appl. No.: 19/653,404

(22) Filed: Apr. 21, 2026

(30) Foreign Application Priority Data
Apr. 28, 2025 (DE) 102025116265.4
May 8, 2025 (DE) 102025117788.0
Oct. 30, 2025 (DE) 102025144485.4

(51) Int. Cl. G01N 35/10; G01N 33/00; G01N 35/00

(52) U.S. Cl. CPC G01N 35/10; G01N 33/007; G01N 33/0073; G01N 35/00623; G01N 35/00712

(57) ABSTRACT

The automatic dispenser according to the present disclosure is able to dispense at least one gas measuring device. The gas measuring device is inserted in a receiving unit of the automatic dispenser. A fluid guidance arrangement connects a housing gas inlet to the receiving unit. In a closing position, a closure closes the receiving unit; in an open position, the closure releases the receiving unit and makes removal possible. In a locking state, a locking unit locks the closure; in a releasing state, the locking unit releases the closure. In response to capturing a user input, a check is made as to whether a usable gas measuring device is inserted in the receiving unit, namely a gas measuring device that can measure a target gas concentration with sufficient accuracy. If a usable gas measuring device is inserted, the closure will be unlocked.

### CROSS-REFERENCE TO RELATED APPLICATIONS

This application claims the priority of German Patent Application No. 102025116265.4, filed on Apr. 28, 2025, and titled “AUTOMATIC DISPENSER FOR GAS MEASURING DEVICES, AND METHOD FOR DISPENSING AND METHOD FOR RETURNING A GAS MEASURING DEVICE”; German Patent Application No. 102025117788.0, filed on May 8, 2025, and titled “AUTOMATIC DISPENSER FOR GAS MEASURING DEVICES, AND METHOD FOR DISPENSING AND METHOD FOR RETURNING A GAS MEASURING DEVICE”; and German Patent Application No. 102025144485.4, filed on Oct. 30, 2025, and titled “AUTOMATIC DISPENSER FOR GAS MEASURING DEVICES, AND METHOD FOR DISPENSING AND METHOD FOR RETURNING A GAS MEASURING DEVICE” each of which is hereby incorporated by reference in its entirety for all nonlimiting purposes.

### TECHNICAL FIELD

The present disclosure relates to an automatic dispenser for gas measuring devices. The present disclosure further relates to a dispensing method for dispensing a gas measuring device using such an automatic dispenser, and to a return method for returning a gas measuring device to such an automatic dispenser.

### BACKGROUND

As is well known, a gas measuring device is configured to measure the concentration of a target gas in a gas sample. A special case of this measurement is that the gas measuring device can automatically decide whether the target gas concentration lies within a specified range or not. The target gas is flammable, combustible, toxic or otherwise harmful to humans, or even essential for human life; it is, for example, oxygen. Or the target gas is an anesthetic. The gas sample originates from a spatial region that is to be monitored for the target gas.

In one application, a user carries such a gas measuring device while he/she is in the spatial region. The portable gas measuring device warns the user if the target gas is present in the spatial region at an impermissible concentration. In another application, a gas measuring device can be set up in a location and transmits a measured target gas concentration to a spatially distant receiver. The present disclosure can be used in combination with both applications.

An automatic dispenser for mobile devices, and in particular for gas measuring devices, is described in EP 3079109 B1 and U.S. Pat. No. 10,409,237 B2. The automatic dispenser described there comprises several storage stations 11 and at least one maintenance station 14. A test gas can be conducted from a gas source 40 via a gas supply line 42 to the maintenance station 14. A gas measuring device 1 can be inserted into and calibrated in the maintenance station 14. After calibration, the gas measuring device 1 is inserted into a storage station 11. A user can enter an output request. Once the output request has been recognized, a group of gas measuring devices is determined depending on a ratio between a maintenance parameter and a maintenance threshold or calibration threshold. A gas measuring device 1 in the group is selected, and the storage station 11, in which the selected gas measuring device 1 is located, is unlocked. If the user wants to return the gas measuring device 1, he/she inserts it in the maintenance station 14, where it is checked.

### SUMMARY

The present disclosure is based on the object of providing an automatic dispenser as well as a dispensing method and a return method that reduce the risk of an error when a gas measuring device is dispensed or returned.

The object is achieved by an automatic dispenser having the features of the claims, by a dispensing method having the features of the claims and by a return method having the features of the claims. Advantageous embodiments are specified in the claims. Advantageous embodiments of the automatic dispenser according to the present disclosure are, where appropriate, also advantageous embodiments of the methods according to the present disclosure, and vice versa.

The automatic dispenser comprises at least one receiving unit, preferably at least two receiving units, particularly preferably at least five receiving units, in particular at least ten receiving units. A gas measuring device can be inserted into the or one receiving unit and can be removed from the receiving unit again. How a gas measuring device operates was described above. An embodiment with one receiving unit is described below; where necessary, an embodiment with several receiving units is described.

The automatic dispenser comprises a housing gas inlet and a fluid guidance arrangement. The fluid guidance arrangement establishes a fluidic connection between the housing gas inlet and a receiving unit, and thus between the housing gas inlet and a gas measuring device that is inserted in the receiving unit. In the case of several receiving units, the fluid guidance arrangement establishes a fluidic connection between the housing gas inlet on the one hand and each receiving unit and thus with each inserted gas measuring device on the other hand.

A closure of the automatic dispenser can be moved back and forth relative to the receiving unit between a closing position and an open position. In the closing position, the closure prevents a gas measuring device from being removed from or inserted into the receiving unit. In the open position, the closure releases the receiving unit and thereby permits this removal and insertion. A locking unit can be moved into a locking state and into a releasing state. The locking unit preferably comprises a locking body that is movable relative to the closure, engages with a receptacle or projection of the closure in the locking state, and does not do so in the releasing state.

While the closure is in the closing position and the locking unit is in the locking state, the locking unit prevents the closure from being moved into the open position. If the locking unit is in the releasing state, the closure is unlocked and thereby released. It can be moved into the open position-either manually or by a positioning (opening) element, e.g. a spring.

In the case of a first and a second receiving unit, the automatic dispenser comprises a first and a second closure as well as a first and a second locking unit. Each closure can be moved independently of the other closure into the closing position and into the open position, and each locking unit can be moved independently of the other locking unit into the locking state and into the releasing state. The first closure and the first locking unit are assigned to the first receiving unit, and the second closure and the second locking unit are assigned to the second receiving unit. The same applies correspondingly to three or more receiving units. In general, to each receiving unit is assigned a closure, and to each closure is assigned a locking unit. Each closure can be moved independently of any other closure from the one position to the other position, and each locking unit can be moved independently of any other locking unit from one state to the other state.

The dispensing method according to the present disclosure is carried out using an automatic dispenser with a receiving unit, a closure and a locking unit. Before the dispensing method is carried out, the following situation is established:
The closure is in the closing position relative to the receiving unit.
The locking unit is in the locking state.

In the case of several receiving units, this situation is established for each receiving unit. In other words, no gas measuring device can be removed from the automatic dispenser before the dispensing method is carried out-optionally with the exception of a service technician with specific access rights.

An input unit of the automatic dispenser can capture a user input. The captured user input includes at least the information that the dispensing of a gas measuring device is desired. Optionally, the user input includes further information, in particular about a desired property or a planned use of a gas measuring device to be dispensed and/or about the person making this user input.

The automatic dispenser is configured to perform the following steps after capturing such a user input, and, according to the dispensing method according to the present disclosure, the capture of the user input triggers the following steps:

A signal-processing control unit of the automatic dispenser checks whether a gas measuring device is inserted in the receiving unit and whether the inserted gas measuring device is usable. A gas measuring device can be used only if intact. In other words, a defective gas measuring device is unusable. A gas measuring device is intact if it can measure the concentration of a given target gas in a gas sample such that the measured target gas concentration does not deviate from the actual target gas concentration by more than a specified absolute or percentage tolerance. This applies to prespecified conditions of use, in particular to a prespecified range of the ambient temperature, ambient humidity and ambient pressure. Embodiments of the present disclosure define optional additional criteria that need to be met for an intact gas measuring device to be usable. It may be that an inserted gas measuring device is intact but currently or for a specific application not usable.

Note: the wording is used that a sensor can measure a physical variable, for example the concentration of a target gas in a gas sample. This wording means that the sensor is configured to measure the physical variable directly, or at least one other variable that correlates with the variable to be measured. Therefore, the or one measured other variable or the combination of the measured other variables together are an indicator for the physical variable to be measured. The measurement provides at least one value for the physical variable sought.

In the case of several receiving units, the control unit searches for a gas measuring device that is inserted in a receiving unit and is usable. During this search, the control unit checks at least once whether an inserted gas measuring device is usable. Optionally, the control unit searches among several inserted gas measuring devices for a usable gas measuring device. If the control unit cannot find a usable gas measuring device, it will preferably cause a corresponding error message to be issued.

It is possible for the control unit to find several inserted and usable gas measuring devices. With this search result, the control unit preferably selects a usable gas measuring device. In one embodiment, the control unit makes this selection depending on how reliably the usable gas measuring devices can measure the target gas concentration, i.e. to what amount the measured target gas concentration deviates from a given and thereby known target gas concentration of a gas sample. For example, the control unit selects the gas measuring device with the smallest deviation. As a rule, each gas measuring device comprises its own power supply unit. It is possible that the control unit makes the selection depending on the current charging state of this power supply unit.

It is also possible for the control unit to trigger the following step if several inserted and usable gas measuring devices are found: for each gas measuring device inserted and usable, one item of information is output on an output unit. The user selects a gas measuring device using the output unit, and the control unit captures this user input. The output unit can be part of the automatic dispenser or part of a mobile device, such as a smartphone. According to a second implementation, a data connection is at least temporarily established between the automatic dispenser and the mobile device.

The finding that an inserted and usable gas measuring device has been found triggers the following further step: the control unit causes the locking unit to be moved into the releasing state. The closure is now unlocked and can be moved into the open position, in particular manually, or it will open by itself. If the automatic dispenser comprises several receiving units, the control unit causes the following for the receiving unit in which the detected gas measuring device is inserted: the locking unit assigned to this receiving unit is moved into the releasing state.

Thanks to the present disclosure, a gas measuring device can only be removed from a receiving unit if the control unit has determined that a usable gas measuring device is inserted in this receiving unit. In particular, this feature prevents the user in many cases from removing and taking a gas measuring device that has already been found to be defective or the state of which is unknown, or which is intact but unusable for a specific application. As long as the closure is closed and locked, it prevents or at least makes it more difficult for an unauthorized person to remove a gas measuring device.

The automatic dispenser according to the present disclosure often requires less space than an automatic dispenser implemented as described in EP 3079109 B1 and U.S. Pat. No. 10,409,237 B2. This advantage is achieved because the automatic dispenser according to the present disclosure comprises only one type of receiving units, wherein the or each receiving unit is configured not only to hold a gas measuring device ready for dispensing but also to allow this gas measuring device to be checked, namely by having the gas measuring device measure a target gas concentration in a supplied gas sample wherein preferably the target gas concentration in the gas sample is known. Of course, the receiving units can vary, in particular with respect to a dimension. The automatic dispenser described in EP 3079109 B1 and U.S. Pat. No. 10,409,237 B2, on the other hand, comprises storage stations in which one gas measuring device is kept ready for dispensing, and at least one maintenance station in which a gas measuring device is calibrated.

According to the present disclosure, the fluid guidance arrangement establishes a fluidic connection between the housing gas inlet and the receiving unit. The automatic dispenser therefore makes it possible for a gas sample to be conducted to an inserted gas measuring device. This gas sample contains the target gas at a known and specified concentration. This makes it possible for the gas measuring device to measure the target gas concentration in the gas sample and for the dispenser to compare the measured target gas concentration with the prespecified target gas concentration. Depending on this comparison, the control unit decides whether the inserted gas measuring device is intact or not. According to the present disclosure, a fluidic connection is established between the housing gas inlet and an inserted gas measuring device. This feature makes it possible for an inserted gas measuring device to measure a target gas concentration in a gas sample, wherein this gas sample has flowed from the housing gas inlet to the inserted gas measuring device. The measurement and comparison can be performed in advance, i.e. before the user desires the dispensing of a gas measuring device.

The decision as to whether a gas measuring device is currently usable or not can be made on the basis of a current measurement and a current comparison. It is not necessary to rely on saved data, which may be outdated. Thanks to the present disclosure, the risk is reduced of the automatic dispenser dispensing a gas measuring device that is currently unable to correctly measure a target gas concentration. A user could be endangered by an incorrect measurement of a target gas concentration. Thanks to the present disclosure, it is not necessary to remove a gas measuring device in order to check it. Rather, the check can often be carried out while the gas measuring device is inserted.

Thanks to the present disclosure, the automatic dispenser according to the present disclosure can simultaneously be used as a test station for the or each inserted gas measuring device. It is possible, but thanks to the present disclosure not necessary, to remove an inserted gas measuring device from the receiving unit in order to check it. Furthermore, thanks to the present disclosure, the following is not necessary: a gas measuring device that is inserted in a receiving unit with a fluidic connection to the housing gas inlet is checked or calibrated and then inserted into another receiving unit where it is held ready for dispensing. Rather, the present disclosure makes it possible to not only check but also to make available a gas measuring device for dispensing in the same receiving unit.

In many cases, an existing test station can be modified such that an automatic dispenser according to the present disclosure is provided after the modification. As a rule, such a test station already comprises at least one receiving unit, the housing gas inlet and fluid guidance arrangement, as well as a control unit. In many cases, it is sufficient to add at least one closure and at least one locking unit and to adapt a software program running on the control unit.

The control unit preferably has read access to a data memory. This data memory contains information for each inserted gas measuring device, indicating whether this gas measuring device is currently intact or not. This information results, for example, from a test procedure in which a test gas with a known target gas concentration is conducted to the inserted gas measuring device and the target gas concentration measured by the gas measuring device is compared with the known target gas concentration. The information may also have been entered by a maintenance technician. The data memory can be part of the automatic dispenser or part of the gas measuring device or can be arranged spatially remote.

According to the present disclosure, a gas measuring device can be removed (taken) from the receiving unit if the closure is in the open position. The closure can be moved into the open position only if the locking unit is in the releasing state. In one embodiment, a user moves the closure to the open position. In another embodiment, the receiving unit comprises an opening element. This opening element can move the closure from the closed into the open position. Preferably, the locking unit secures the closed closure against the force of the opening element. This embodiment has the following effect: as soon as the locking unit is moved into the releasing state, i.e. unlocked, the opening element automatically opens the closure. The closure thus springs open by itself. This embodiment eliminates the need to indicate to a user on a separate display element that a usable gas measuring device can now be removed from the receiving unit. Furthermore, it is not necessary for the user to open the closure manually.

In the case of several receiving units, preferably each receiving unit comprises a respective opening element that can move the associated closure to the open position. This embodiment also eliminates the need to indicate to a user which receiving unit contains a usable gas measuring device that can now be removed.

Preferably, the or each opening element is implemented as a purely mechanical component, thus requiring no supply of electrical, pneumatic, or hydraulic energy and no actuation. In particular, the or each opening element comprises a spring.

It is possible that the closure can be moved, i.e. displaced, from one position to another by a linear movement relative to the receiving unit. In a preferred embodiment, however, the closure can be moved from the one position to the other by a rotation, i.e. can be rotated. Preferably the force of gravity tends to rotate the closure to the closing position. In the case of several receiving units and thus several closures, these closures are preferably arranged next to one another such that all closures can be rotated about the same axis of rotation, independently of one another.

An embodiment was described above in which an opening element can move the closure to the open position. Preferably the closure is rotatably arranged, and the opening element can rotate the closure to the open position, preferably against the force of gravity.

In many cases, a gas measuring device comprises its own power supply unit, in particular at least one rechargeable battery. In one embodiment, the receiving unit comprises an electrical contact. In the case of several receiving units, each receiving unit preferably comprises an electrical contact.

In one implementation, the power supply unit is charged or can be charged as long as the gas measuring device is inserted in the receiving unit. Charging is carried out with the aid of the electrical contact. The automatic dispenser is connected to a stationary power supply network and/or comprises its own power supply unit. Thanks to this implementation, it is not necessary to remove the gas measuring device in order to recharge its power supply unit. This saves time compared to an embodiment in which the gas measuring device would have to be removed from the dispenser and inserted into a separate charging station.

The automatic dispenser preferably comprises a voltage sensor. If a gas measuring device is inserted in the receiving unit, an electrical connection between the voltage sensor and the power supply unit of the inserted gas measuring device is established or can be established by means of the electrical contact. The voltage sensor can measure the current charging state of the power supply unit. It is also possible that the dispenser can capture the current charging state of the power supply unit from a voltage sensor being part of the gas measuring device.

In the case of several receiving units, the following alternatives are possible: in one alternative, all receiving units are assigned the same voltage sensor. In another alternative, each receiving unit is assigned its own voltage sensor.

According to the present disclosure, the control unit checks whether an inserted gas measuring device is usable. According to the embodiment with the electrical contact, the control unit decides that the inserted gas measuring device is currently not usable if the measured charging state is less than a charging state threshold. In one implementation, this charging state threshold is fixed (given in advance). A different implementation will be described below.

According to the present disclosure, the input unit can capture a user input. In one embodiment, this user input also includes a specification as to how long a use of a gas measuring device to be dispensed is intended. During this use, it is usually not possible or at least not desirable to charge or to exchange the power supply unit. The control unit is configured to predict, depending on the measured charging state, how long the power supply unit of the inserted gas measuring device will be able to supply the gas measuring device with electrical energy.

According to this embodiment, the control unit assesses the inserted gas measuring device as usable if it is intact and if its power supply unit can supply the gas measuring device with electrical energy for at least the captured and desired duration. The charging state threshold thus depends on the desired duration of use.

To make the prediction, the control unit uses a signal from the voltage sensor, i.e. the measured current charging state, as well as a specified or pre-measured value for how much electrical energy the gas measuring device consumes per unit of time.

Thanks to this embodiment, in many cases an inserted gas measuring device is more frequently considered to be usable than if only a fully charged gas measuring device were usable.

According to the embodiment just described, the control unit considers an inserted gas measuring device to be usable only if it is intact and if the measured charging state of its power supply unit is above a prespecified (given) charging state threshold or a threshold that depends on the user input. In an implementation of this embodiment, the control unit is configured as follows: If the measured charging state is lower than the charging state threshold, the control unit predicts a time at which the charging state will reach the charging state threshold. With this implementation, the power supply unit is charged while the gas measuring device is inserted. To make this prediction, the control unit uses the measured current charging state as well as a prespecified or pre-measured value for how quickly the charging state increases during charging. This value may depend on the type and/or age of the gas measuring device. Preferably, the predicted time span required for charging is output by the automatic dispenser in a form perceptible to a human.

Preferably, the automatic dispenser comprises several receiving units. It is possible for the control unit to find several inserted and usable gas measuring devices. In one embodiment, the control unit is configured to select that gas measuring device whose power supply unit has the highest charging state or can supply the gas measuring device with electrical energy for the longest time. Or the control unit is configured to apply a selection criterion that depends on the accuracy of the target gas measurement and on the measured charging state. The control unit ensures that the gas measuring device selected in this way can be removed (taken) from the automatic dispenser.

In one implementation, the voltage sensor is also used to detect an error. The voltage sensor repeatedly measures the current charging state of an inserted gas measuring device, optionally of several gas measuring devices, and the control unit derives a temporal course of the charging state. If this charging state remains unchanged for a sufficiently long time span, this can in particular be due to one of the following three causes:
The power supply unit is fully charged (a desired situation).
The gas measuring device is not correctly inserted in the receiving unit, and for this reason the power supply unit is not electrically contacted (an error).
The power supply unit is defective, or the automatic dispenser is unable to supply the power supply unit with electrical energy (an error).

According to the present disclosure, the input unit can capture a user input. In one embodiment, the captured user input additionally includes a specification about a desired property of a gas measuring device. Examples of a desired property are the following:
The gas measuring device should be able to measure a specific target gas, optionally several target gases that may occur simultaneously.
The gas measuring device should be able to reliably measure the concentration of the target gas under a prespecified environmental condition.
The prespecified environmental condition defines, for example, a range for the ambient temperature or for the ambient humidity or the ambient pressure. For example, the gas measuring device should be able to measure the target gas concentration even at a very high ambient temperature or a very high ambient humidity or in a tube.
The gas measuring device should not be heavier and/or larger than a prespecified upper threshold for its weight or for one dimension.
The gas measuring device should be able to issue an alarm in a specific way, for example haptically (through vibrations).
The gas measuring device should have an illuminated display area so that a display can be read even in the dark.
The gas measuring device should be able to capture or generate voice inputs and/or outputs in a specific language.

According to the embodiment just described, the control unit considers a gas measuring device to be usable only if it is intact and also has the or every desired property. Only if it has the or every desired property the locking unit will be moved into the releasing state, thus allowing the gas measuring device to be removed. This embodiment prevents the following undesirable situation: a user takes a gas measuring device from the automatic dispenser that is intact but cannot be used for the intended purpose at all, or not with sufficient reliability or ergonomics.

Preferably, the captured user input additionally comprises an identification of the user who wants to take a gas measuring device from the automatic dispenser. The control unit of the automatic dispenser uses this identification to check whether the user is authorized to take a gas measuring device. The control unit checks, in particular, whether the user is authorized at all and/or qualified to use a gas measuring device having a specific desired property. The identification can, for example, comprise a username and a password. It is possible for the input unit to comprise a reader device that can read a chip on a user's chip card, or a sensor for a user's biometric feature.

The implementation described below of this embodiment can be combined with the embodiment already described, in which the captured user input specifies a desired property of a gas measuring device. According to this development, a user must possess a certain qualification to be authorized to use a gas measuring device with this captured desired property. The respective qualification profile of a set of registered users is stored in a data memory. After the input unit has captured a user identification, the control unit determines the stored qualification profile of that user. The control unit considers a gas measuring device to be usable only if it is intact and if the registered and recognized user is authorized to use the gas measuring device according to the determined qualification profile.

The two embodiments just described can be combined with one another. The control unit considers an inserted gas measuring device according to this combination to be usable only if it has the or every property required or desired according to the user input, and if, in addition, the registered and recognized user is authorized to use this gas measuring device. It is possible for a user's qualification profile to specify which property a gas measuring device has or can have that this user is authorized to use it.

According to the present disclosure, the fluid guidance arrangement establishes a fluidic connection between the housing gas inlet and an inserted gas measuring device. In one embodiment, the receiving unit comprises a contact element. The contact element can be moved back and forth between a contacting and a spaced-apart position relative to an inserted gas measuring device. According to this embodiment, the fluidic connection with the housing gas inlet just described is established only if the contact element is in the contacting position, wherein the fluidic connection leads through the contact element. Otherwise, this fluidic connection is interrupted. In the case of several receiving units, each receiving unit preferably comprises a contact element, and each contact element can be moved from one position to another independently of the or of any other contact element.

One advantage of the movable contact element compared to an embodiment without a contact element is the following: generally, the inserted gas measuring device can be removed from the receiving unit and reinserted only, or at least more easily, if the contact element is in the spaced-apart position.

In one embodiment, the contact element comprises a valve. The fluidic connection is established while the valve is open. The process of moving the contact element to the contacting position preferably triggers the process of opening the valve. Conversely, the process of moving the contact element out of the contacting position preferably triggers the process of closing the valve.

Preferably, the contact element is located between the closure and a region of the receiving unit, wherein the region is, for example, a recess into which a gas measuring device can be inserted. If a gas measuring device is inserted in the receiving unit and the closure is in the closing position, the contact element will be located between the closure and the gas measuring device.

In a preferred implementation, both the closure and the contact element are rotatably arranged relative to the receiving unit. The two axes of rotation of this rotatable arrangement are preferably parallel to one another or coincide with one another.

Particularly preferably, the contact element is mechanically coupled to the closure such that the following effect is achieved: a movement of the closure from the open position to the closing position causes the contact element to be moved from the spaced-apart position to the contacting position. Thanks to this implementation, only a single action is required to ensure that the closure seals the receiving unit and that a fluidic connection is established between the housing gas inlet and a measuring chamber of the inserted gas measuring device. In particular, a user who has inserted the gas measuring device need only perform one action.

In one embodiment, an actuating element of the automatic dispenser can be operated manually. The actuating element is connected to the locking unit such that the following chain of effects is realized: an actuation of the actuating element causes the locking unit to be moved into the releasing state. In the case of several receiving units and hence several locking units, the following alternative implementations are possible:
The same actuating element is connected to all locking units such that by actuating the actuating element, all locking units are moved into the releasing state.
Each locking unit is connected to an actuating element. Each actuating element can be operated independently of the or of any other actuating element. A locking unit is moved into the releasing state by actuating the connected actuating element. This allows each locking unit to be moved into the releasing state independently of the or of any other locking unit.

In particular, the embodiment with the actuating element makes it possible, for a maintenance technician, to move the locking unit to the releasing state and open the closure, regardless of the state of the inserted gas measuring device. In particular, the maintenance technician is enabled to remove a defective gas measuring device. In one implementation, a person must be authorized before activating the actuating element.

The actuating element is preferably a purely mechanical part. “Purely mechanical” means that the actuating element requires no electrical, pneumatic, or hydraulic energy, nor any automatic control input. With this embodiment, the closure can usually be opened even if a defect has occurred or if the automatic dispenser's electrical power supply has failed or has been interrupted. For example, the actuating element comprises a lever and/or a wheel.

In one implementation, the mechanical actuating element comprises a mechanical lock and a matching key. The mechanical lock is embedded in the closure or the locking unit. The mechanical lock can selectively be moved into a closing state and into an opening state with the aid of the matching key. The mechanical lock is connected to the associated locking unit as follows, preferably purely mechanically: moving the mechanical lock into the opening state causes the locking unit to be moved into the releasing state. Compared to a lever or a wheel, the embodiment with the mechanical lock and the key reduces the risk of an unauthorized person opening the closure and removing an inserted gas measuring device.

In a further development of this implementation, the mechanical lock is coupled to the locking unit as follows: moving the locking unit to the locking state causes the mechanical lock to be moved into the closing state. Or conversely, moving the mechanical lock to the closing state causes the locking unit to be moved into the locking state. These two effects occur at least while the associated closure is in the closing position.

It is also possible for the actuating element to comprise a user's reader device with which a user can authorize himself/herself. The user's reader device captures, for example, a password or a biometric feature of the user. Upon successful authorization, the or each locking unit is automatically moved into the releasing state.

The situation could occur in which a user attempts to open a closure of a receiving unit, even though the locking unit for this closure is in the locking state. In one embodiment, the receiving unit additionally comprises a sensor, for example a force sensor or a position sensor. This sensor is configured to detect an event in which an attempt is being made to open the locked closure. If the sensor detects this event, the control unit will cause an alarm to be issued.

In one embodiment, the automatic dispenser comprises a camera and a signal-processing image evaluation unit. The camera can produce an image of a gas measuring device. In one implementation, the camera is positioned such that the following is made possible: the camera can produce an image of a gas measuring device that is inserted in a receiving unit. In a preferred implementation, however, the camera is positioned such that the following is made possible: the camera can produce an image of a gas measuring device that is located outside the automatic dispenser. Preferably, the automatic dispenser comprises a base, for example a tray or plate, and the camera can produce an image of a gas measuring device on the base. The image evaluation unit can evaluate the image or images of a gas measuring device and thereby automatically decide whether the shown gas measuring device has any visually detectable damage or not, and preferably, if damage is found, which type of damage. The visually detectable damage is, in particular, mechanical damage or pollution.

The embodiment with the camera and the image evaluation unit makes it possible to automatically check whether a dispensed gas measuring device is damaged. If the camera is configured and positioned to produce an image of a gas measuring device outside the automatic dispenser, it will be possible to check whether the gas measuring device is damaged in a visually detectable way before it is returned.

The present disclosure further relates to a system. This system comprises an automatic dispenser according to the present disclosure, a gas measuring device, a fluid guidance unit, and a container with a test gas. The gas measuring device is inserted in the receiving unit. In the case of several receiving units, the gas measuring device is inserted in one receiving unit, and preferably a further gas measuring device is inserted in a further receiving unit. A measuring chamber of the or of each gas measuring device is configured to receive a gas sample, and the gas measuring device measures the concentration of a target gas in the received gas sample.

The test gas in the container contains a target gas with a specified target gas concentration. The fluid guidance unit can establish a fluidic connection between the container with the test gas and the housing gas inlet of the automatic dispenser. In one embodiment of the system, the fluid guidance unit is permanently connected to the automatic dispenser; in another embodiment, it is detachably connected. The fluid guidance arrangement connects the housing gas inlet to the measuring chamber of the inserted gas measuring device. In one implementation, this fluidic connection is established only if the above-described movable contact element of the receiving unit is in the contacting position.

The system according to the present disclosure is configured to perform the following steps:
The system causes or enables a gas sample containing the test gas to flow from the container to the receiving unit and then to the inserted gas measuring device.
The control unit causes the inserted gas measuring device to measure the target gas concentration in the gas sample.
The measured target gas concentration is compared with the prespecified target gas concentration, preferably by the dispenser.
The control unit decides that the gas measuring device is not intact if the measured target gas concentration deviates from the prespecified target gas concentration by more than the tolerance.

In one embodiment, the system is configured to perform a test method at least once. The test method is preferably performed repeatedly, for example at a prespecified frequency or every time the automatic dispenser is switched on. It is also possible for the test method to be performed in response to a corresponding captured user input. During the or each performance of the test method, the steps just described are carried out, and the control unit decides whether the inserted gas measuring device is intact or not. If there are several receiving units, the control unit decides for the or each inserted gas measuring device whether it is intact or not. The control unit preferably saves the respective result of each test and uses the saved result if the dispensing of a gas measuring device is requested.

In one embodiment, in the case of several receiving units, the test method is carried out for each gas measuring device that is inserted in a receiving unit. In another embodiment, at least one receiving unit is selected beforehand, for example in response to a corresponding user input or automatically depending on a time point at which the gas measuring device in this receiving unit was last checked. The test method is carried out for the or each gas measuring device that is inserted in a selected receiving unit.

The embodiment just described makes it possible to check how accurately a gas measuring device can measure the concentration of a target gas. In one embodiment, the system comprises two containers with two different test gases, wherein the respective concentration of each test gas in the container is known. A pneumatic switch with two inputs and one output can be actuated and, depending on the actuation, conducts test gas from one container or test gas from the other container to the automatic dispenser. This embodiment makes it possible to check with what accuracy the gas measuring device is able to measure the respective concentration of the two target gases. This embodiment can be extended to more than two target gases.

In one embodiment, the input unit of the automatic dispenser is configured to capture a user input. This user input specifies that a gas measuring device that has been inserted into the receiving unit is to be checked, namely by the test method just described. In the case of several receiving units, the user input can specify at least one receiving unit or specify that all inserted gas measuring devices should be checked.

The present disclosure further relates to a return method for returning a gas measuring device to an automatic dispenser according to the present disclosure. According to the return method, the following situation is created before the steps of the return method are carried out:
There is no gas measuring device inserted in the receiving unit. In the case of several receiving units, at least one receiving unit does not have a gas measuring device inserted.
The or each closure is in the closing position.
The or each locking unit is in the locking state.

A reader device in the automatic dispenser captures a machine-readable identifier of the gas measuring device that is to be returned. For example, the reader device reads a data memory, in particular an NFC chip or RFID chip, of the gas measuring device, or captures a barcode or a QR code or an alphanumeric identifier on the outside of the gas measuring device.

The step of the identifier having been captured triggers the following step: the control unit checks whether the receiving unit can accommodate the gas measuring device or not. The gas measuring device is the device whose identifier has been captured. For example, the captured identifier is associated with at least one dimension and/or an identification of an electrical interface, and the control unit checks, depending on the captured dimension and/or identification of the interface, whether the receiving unit is suitable or not.

In the case of several receiving units, the control unit searches for a receiving unit into which no gas measuring device has currently been inserted and which can accommodate the gas measuring device.

The determination that the or one receiving unit can accommodate the gas measuring device triggers the following step: the control unit causes the locking unit to be moved into the releasing state for the suitable receiving unit. Now the closure can be opened or springs open by itself, and the gas measuring device can be inserted into the receiving unit. In one implementation, the gas measuring device can be inserted after the contact element has been moved into the spaced-apart position.

This return method reduces the risk of the gas measuring device being inserted into the automatic dispenser arbitrarily, and in particular reduces the risk of the gas measuring device being inserted into an unsuitable receiving unit. The return method does not necessarily require a user wishing to return the gas measuring device to authorize himself/herself. Furthermore, this return method makes it easier in many cases for the automatic dispenser to check the returned gas measuring device.

In one embodiment of the return method, the step of the gas measuring device having been inserted into the or a receiving unit triggers the following further steps: a gas sample from the aforementioned container with test gas is conducted to the gas measuring device that has just been inserted. The gas measuring device measures the target gas concentration, and the measured value is compared with the given target gas concentration. Thanks to this embodiment, the gas measuring device is checked immediately after being returned.

In the case of several receiving units, the automatic dispenser preferably comprises one valve for each receiving unit. The valve for a receiving unit is opened while a gas measuring device inserted in this receiving unit is to be checked. Otherwise, the valve is closed. This implementation makes it possible to specifically check a particular gas measuring device, for example one that has just been inserted. This implementation saves test gas compared to an embodiment in which all inserted gas measuring devices are checked during each test.

### BRIEF DESCRIPTION OF THE DRAWINGS

The present disclosure will be described below on the basis of an exemplary embodiment. In the drawings:

FIG. 1 shows a top view of a test station with three receiving units;

FIG. 2 shows a side view of the test station in FIG. 1;

FIG. 3 shows a schematic top view of an automatic dispenser according to the present disclosure;

FIG. 4 shows, in a view obliquely from above, a first implementation of an automatic dispenser according to the present disclosure;

FIG. 5 shows, in a view from above, a region of the automatic dispenser in FIG. 4;

FIG. 6 shows, in a view obliquely from above, a part of the region in FIG. 5;

FIG. 7 shows a side view of the automatic dispenser in FIG. 4;

FIG. 8 shows, in a view obliquely from above, a second implementation of an automatic dispenser according to the present disclosure, wherein the protective flap is open;

FIG. 9 shows the automatic dispenser in FIG. 8 with the protective flap closed;

FIG. 10 shows, in a view obliquely from behind, the automatic dispenser in FIG. 9; and

FIG. 11 shows a single receiving unit of the automatic dispenser in FIG. 8 to FIG. 10 with the closure open.

### DETAILED DESCRIPTION

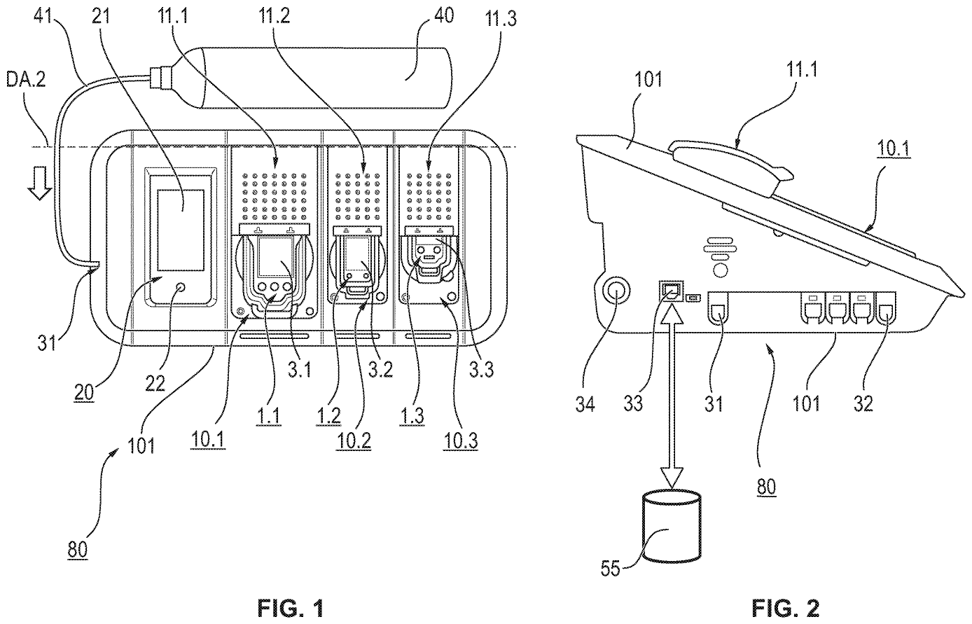

FIG. 1 and FIG. 2 show a top view and side view, respectively, of a test station 80. The test station 80 comprises three receiving units 10.1, 10.2, 10.3, which are arranged next to one another from left to right in a housing 101.

Each receiving unit 10.1, 10.2, 10.3 accommodates one gas measuring device 1.1, 1.2, 1.3 in a recess. In the example shown, the three receiving units 10.1, 10.2, 10.3 are of different sizes. However, this is not absolutely necessary. The test station 80 can also comprise a different number of receiving units.

Each gas measuring device 1.1, 1.2, 1.3 comprises its own power supply unit and can visually display the result of a measurement on a display unit 3.1, 3.2, 3.3. In one embodiment, the display unit 3.1, 3.2, 3.3 comprises a backlight. Each gas measuring device 1.1, 1.2, 1.3 further comprises operating elements 4.1, 4.2, 4.3.

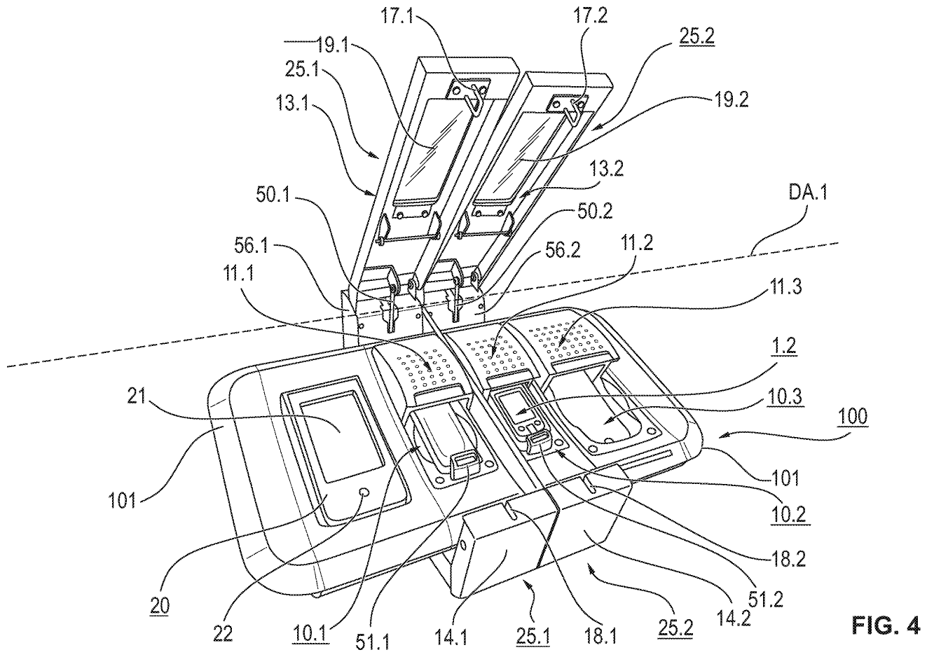

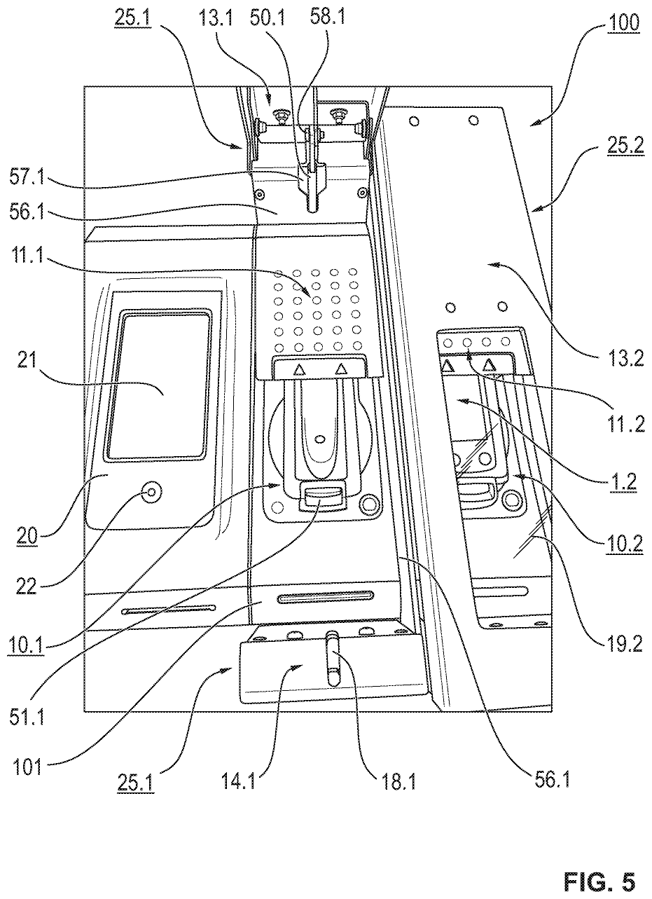

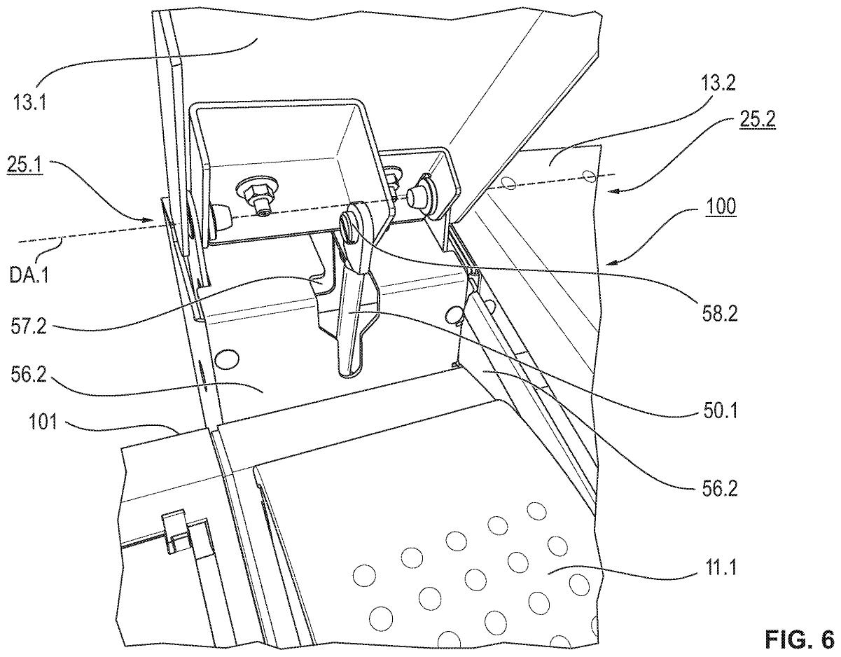

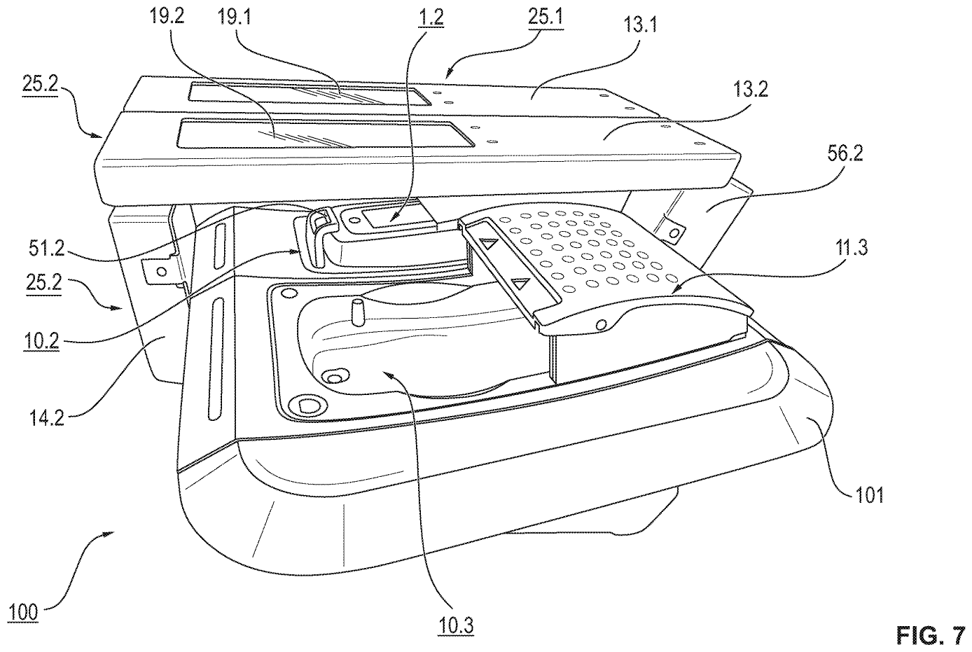

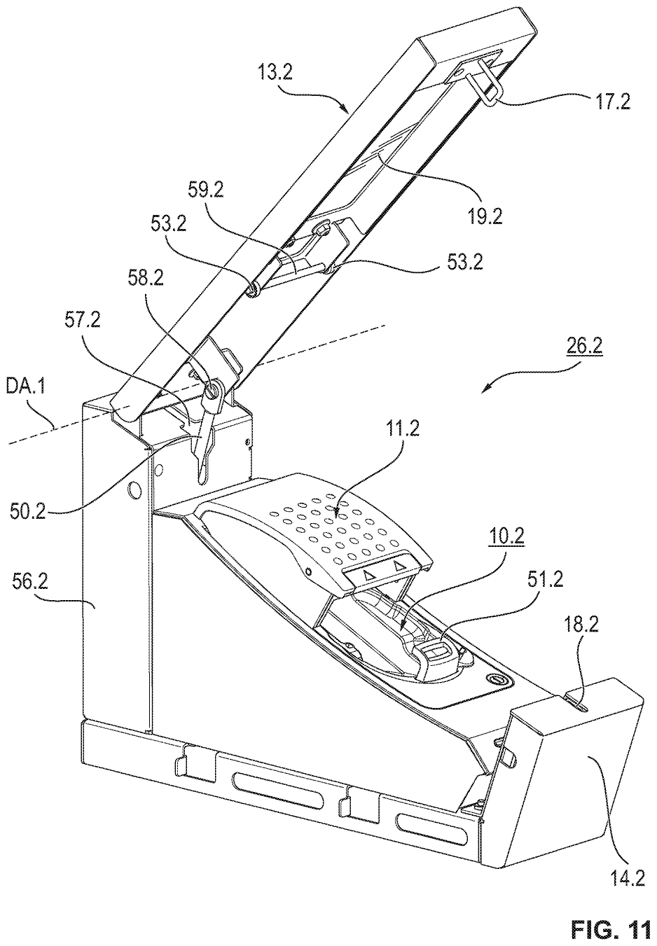

Each receiving unit 10.1, 10.2, 10.3 comprises one electrical contact 51.1, 51.2, 51.3, cf. FIG. 4, FIG. 5, FIG. 7 and FIG. 11. As long as a gas measuring device 1.1, 1.2, 1.3 is inserted in a receiving unit 10.1, 10.2, 10.3, the power supply unit of this gas measuring device 1.1, 1.2, 1.3 will be charged by means of the electrical contact 51.1, 51.2, 51.3 or alternatively by inductive charging.

Preferably, each receiving unit 10.1, 10.2, 10.3 comprises one device sensor (not shown). This device sensor can determine whether there is a gas measuring device 1.1, 1.2, 1.3 currently inserted in the receiving unit 10.1, 10.2, 10.3 or not. In one implementation, the device sensor comprises a contact switch. It is also possible for the device sensor to detect whether a current is flowing through the electrical contacts 51.1, 51.2, 51.3, which is only possible while a gas measuring device is inserted.

Each receiving unit 10.1, 10.2, 10.3 comprises one contact element 11.1, 11.2, 11.3. A gas sample can be conducted through this contact element 11.1, 11.2, 11.3 to a gas inlet 2.1, 2.2, 2.3 of an accommodated gas measuring device 1.1, 1.2, 1.3. The three contact elements 11.1, 11.2, 11.3 are rotatable independently of one another relative to the housing 101 about a common axis of rotation DA.2. FIG. 1 shows the three contact elements 11.1, 11.2, 11.3 in a downward-rotated position, in which they cover the respective gas inlet 2.1, 2.2, 2.3 of an accommodated gas measuring device 1.1, 1.2, 1.3. If a contact element 11.1, 11.2, 11.3 is rotated upward, an inserted gas measuring device 1.1, 1.2, 1.3 can be removed from the receiving unit 10.1, 10.2, 10.3, and the same or a different gas measuring device can be inserted. The downward-rotated position is the contacting position, and the upward-rotated position is the spaced-apart position.

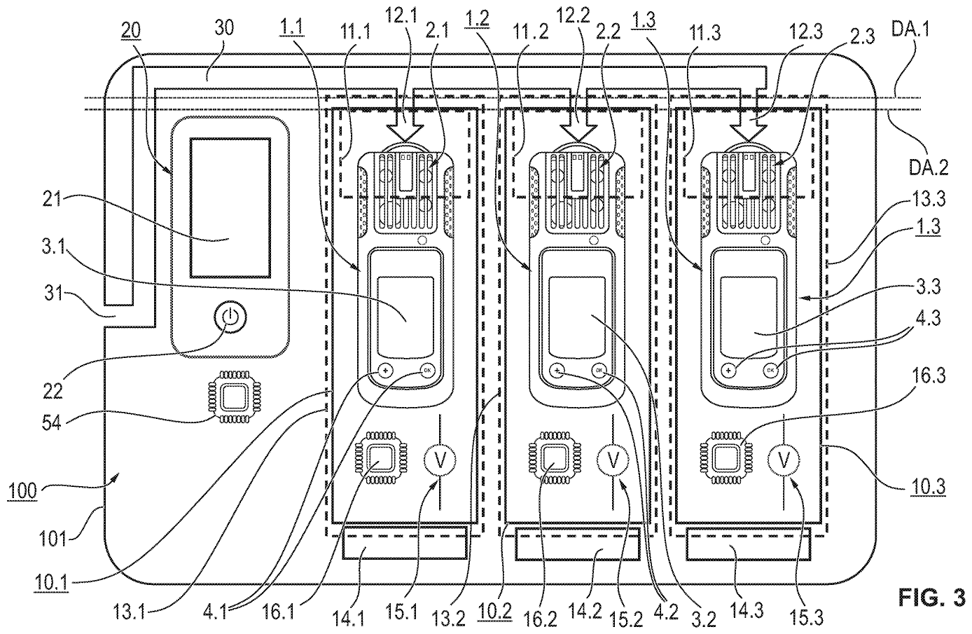

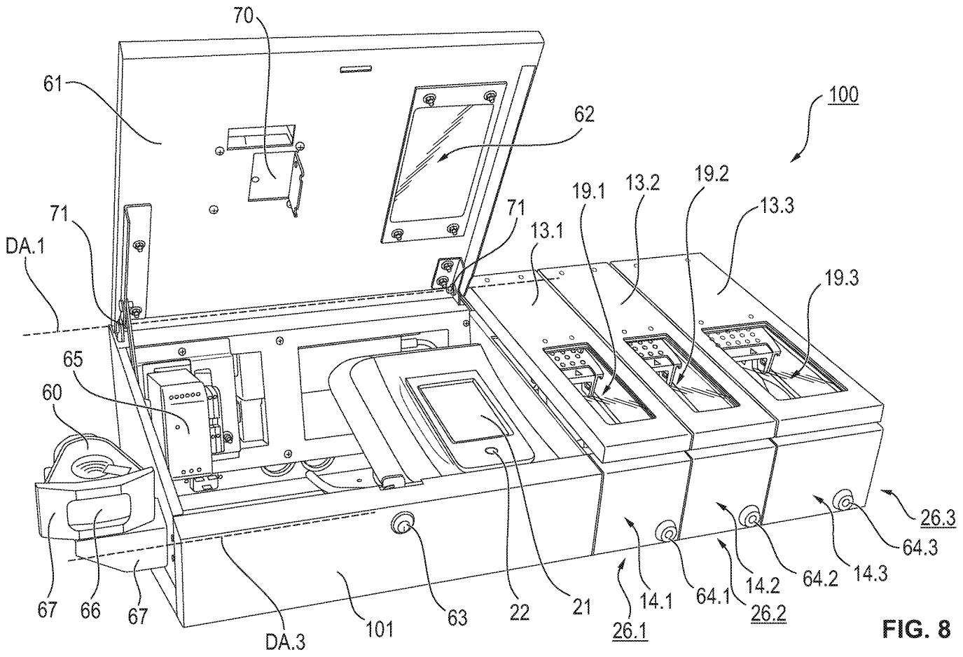

FIG. 3 shows a schematic top view of an automatic dispenser 100 according to the present disclosure, which comprises the test station 80 in FIG. 1 and FIG. 2. FIG. 4 to FIG. 7 show a first implementation in which the test station 80 in FIG. 1 and FIG. 2 is supplemented by at least one closing component, thus providing an automatic dispenser 100 according to the present disclosure. In the example shown, a closing component 25.1, 25.2 is mounted around each of the two receiving units 10.1 and 10.2, while no closing component is mounted around the receiving unit 10.3. FIG. 8 to FIG. 11 show a second implementation in which the automatic dispenser 100 comprises three receiving modules 26.1, 26.2, 26.3. The same reference signs have the same meanings.

FIG. 1 shows a container 40 in the form of a bottle. The container 40 receives a test gas, preferably under overpressure. This test gas comprises at least one target gas with a known concentration. A line 41 conducts the test gas from the container 40 to a housing gas inlet 31 in the housing 101. The line 41 functions as the external fluid guidance unit-namely, externally to the automatic dispenser 100. A gas line 30 connects the housing gas inlet 31 to gas supply elements 12.1, 12.2, 12.3 in the contact elements 11.1, 11.2, 11.3, cf. FIG. 3. The gas line 30 and the contact elements 11.1, 11.2, 11.3 with the gas supply elements 12.1, 12.2, 12.3 belong to the fluid guidance arrangement in the exemplary embodiment. Preferably, a controllable valve or other pneumatic control element is located inside a contact element 11.1, 11.2, 11.3. Depending on the actuation, the valve either opens or blocks the gas supply element 12.1, 12.2, 12.3. Preferably a volume flow sensor or pressure sensor (not shown) is used for detecting a leak in the fluid guidance arrangement.

As long as the container 40 is connected to the housing gas inlet 31 by means of the line 41, the test gas from the container 40 will be conducted through the lines 41 and 30 to the gas supply elements 12.1, 12.2, 12.3. In one embodiment, the container 40 is permanently connected to the test station 80 and thus to the automatic dispenser 100 during operation of the test station 80. Optionally, a cap (not shown) can close the container 40.

A display and operating unit 20 comprises a screen 21, preferably a touchscreen, and a power button 22, cf. FIG. 1. A user can enter user inputs with the aid of the screen 21. In the first implementation, the screen 21 and the power button 22 are accessible to any user, cf. FIG. 3 to FIG. 5. The automatic dispenser 100 according to the second implementation comprises a protective flap 61, which is attached to the housing 101 by means of a hinge 71 and can be rotated relative to the housing 101 about the same axis of rotation DA. 1 as the closures 13.1, 13.2, 13.3. The folded-down protective flap 61 can be locked and preferably also opened again with the aid of a mechanical lock 63 in the housing 101 and a matching key, in a similar way to a cash box. Instead of or in addition to the mechanical lock 63, an electronic lock (not shown) may also be present.

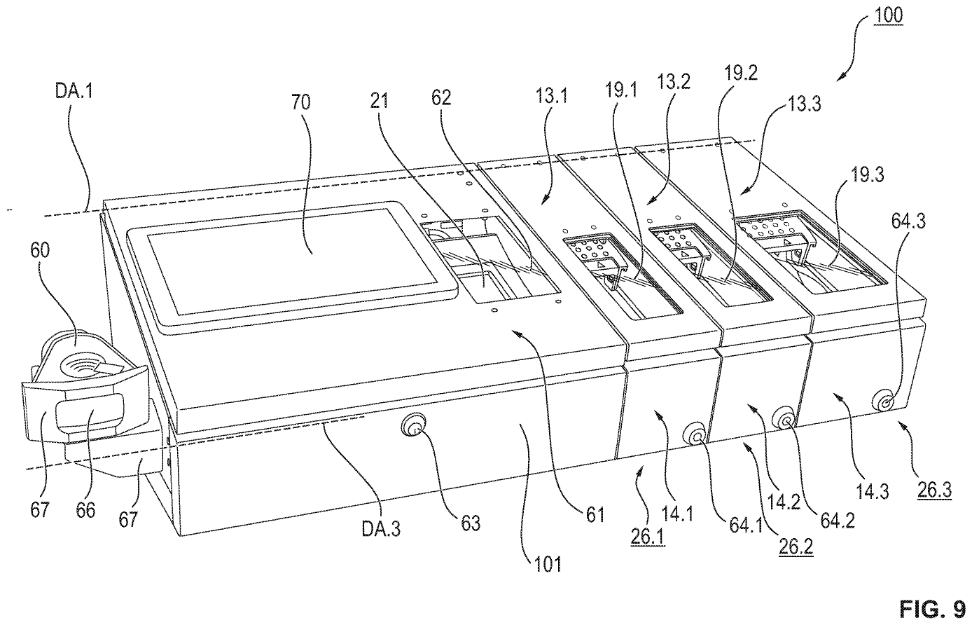

A touchscreen 70 is embedded in the protective flap 61, cf. FIG. 9. With the protective flap 61 closed, a user enters user inputs on the screen 70. While the protective flap 61 is closed, the screen 21 is visible through a window 62 and can be operated only with the protective flap 61 open but not if the protective flap 61 is closed. The screen 21 is intended to be used by a maintenance technician. Accordingly, outputs to a user are output on the screen 70, for example the information that a gas measuring device with the required or desired properties is currently unavailable. However, outputs to a maintenance technician, for example information about the current state of an inserted gas measuring device 1.1, 1.2, 1.3 or of the automatic dispenser 100, are output on the screen 21.

As long as a gas measuring device 1.1, 1.2, 1.3 is inserted in a receiving unit 10.1, 10.2, 10.3 and the container 40 is connected, a gas sample of the test gas will be conducted through the gas inlet 2.1, 2.2, 2.3 and into the measuring chamber of the gas measuring device 1.1, 1.2, 1.3. The sensor of the gas measuring device 1.1, 1.2, 1.3 measures the target gas concentration in the gas sample in the measuring chamber. The display and operating unit 20 and/or the separate display unit 3.1, 3.2, 3.3 shows the result of the measurement for each inserted gas measuring device 1.1, 1.2, 1.3, i.e. with three inserted gas measuring devices 1.1, 1.2, 1.3, three measurements are displayed. Optionally, the given concentration of the target gas in the gas sample is also displayed, wherein this gas sample comes from the test gas in container 40. Of course, it is possible to use several containers with different test gases one after the other.

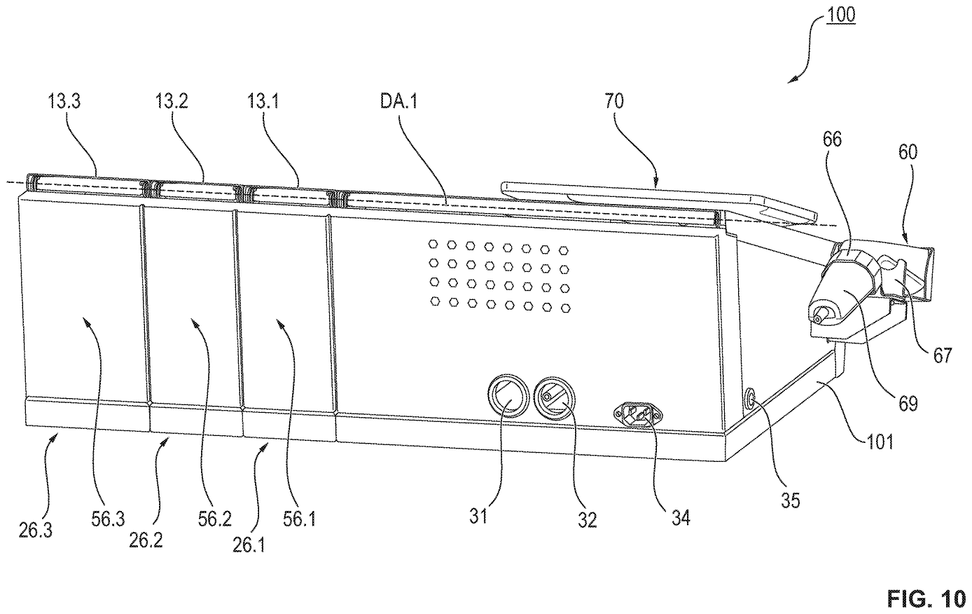

The side view in FIG. 2 and FIG. 10 shows the housing gas inlet 31, a further housing gas inlet 32, a socket 35 for a scanner cable and a socket 34. A gas line (not shown) connects the further housing gas inlet 32 to the gas line 30 or to the gas supply elements 12.1, 12.2, 12.3. A plug can be inserted into the socket 34 to connect the test station 80 and the automatic dispenser 100 to a stationary power supply network and thus supply them with electrical energy. Optionally, the test station 80 and the automatic dispenser 100 comprise their own power supply unit, preferably a rechargeable power supply unit.

In the exemplary embodiment, the test station 80 and the automatic dispenser 100 are connected via an Ethernet cable to a database 55 shown schematically. The Ethernet cable is plugged into a port 33, cf. FIG. 2. In the database 55, a data set is created for each gas measuring device of a plurality of gas measuring devices, including for the gas measuring devices 1.1, 1.2, 1.3. The data set for a gas measuring device 1.1, 1.2, 1.3 comprises a unique identifier, an item of information about the type, and an item of information about the current status of the gas measuring device 1.1, 1.2, 1.3. The item of information about the type includes an identification indicating which target gas or gases the gas measuring device 1.1, 1.2, 1.3 can measure. The item of information about the current status includes an identification of whether the gas measuring device 1.1, 1.2, 1.3 is currently inserted in a receiving unit 10.1, 10.2, 10.3 of the automatic dispenser 100, preferably an item of information about into which receiving unit it is inserted, or whether the gas measuring device 1.1, 1.2, 1.3 is currently in use or is undergoing repair. In addition, the status information preferably includes an identification of whether the gas measuring device 1.1, 1.2, 1.3 was classified as intact or as defective during the last test.

The automatic dispenser 100 comprises a signal-processing control device 54, cf. FIG. 3, and, for each receiving unit 10.1, 10.2, 10.3:
a closure 13.1, 13.2, 13.3,
a locking unit 14.1, 14.2, 14.3 for the closure 13.1, 13.2, 13.3,
a voltage sensor 15.1, 15.2, 15.3, and
a signal-processing evaluation unit 16.1, 16.2, 16.3.

Alternatively, a control unit 65 performs not only the functions of the control device 54 but also the functions of the evaluation units 16.1, 16.2, 16.3, cf. FIG. 8. The designation “the control unit 16.1, 16.2, 16.3, 54, 65” is used hereinafter.

The voltage sensor 15.1, 15.2, 15.3 of a receiving unit 10.1, 10.2, 10.3 measures the current charging state of the power supply unit of an inserted gas measuring device 1.1, 1.2, 1.3.

Each closure 13.1, 13.2, 13.3 is rotatable relative to the housing 101 about an axis of rotation DA.1 and is in each case assigned to a receiving unit 10.1, 10.2, 10.3. The axis of rotation DA.1 is parallel to the axis of rotation DA.2, about which the contact elements 11.1, 11.2, 11.3 are rotatable. The two axes of rotation DA.1 and DA.2 lie in the drawing plane of FIG. 1 and FIG. 3 and are perpendicular to the drawing plane of FIG. 2. In the schematic illustration in FIG. 3, and in FIG. 8 to FIG. 10, the closures 13.1, 13.2, 13.3 are lowered and are therefore in the closing position. If the closure 13.1, 13.2, 13.3 is lowered, the contact element 11.1, 11.2, 11.3 of a receiving unit 10.1, 10.2, 10.3 will be located between the closure 13.1, 13.2, 13.3 and a gas measuring device 1.1, 1.2, 1.3 inserted in the receiving unit 10.1, 10.2, 10.3 or the empty recess. In the illustration in FIG. 4, the two closures 13.1 and 13.2 are rotated upward (open), and the closure 13.3 is not shown. All three contact elements 11.1, 11.2, 11.3 are rotated downward. A gas measuring device 1.2 is inserted in the receiving unit 10.2, while there is no gas measuring device inserted in the other two receiving units 10.1 and 10.3. In FIG. 5, the closure 13.1 is rotated upward and closure 13.2 downward. In the side view in FIG. 7, the two closures 13.1 and 13.2 are rotated downward.

Each closure 13.1, 13.2, 13.3 comprises
a rectangular frame, which in the exemplary embodiment is made of metal,
a window 19.1, 19.2, 19.3, and
a projection 17.1, 17.2, 17.3, which points towards the housing 101.

If the closure 13.1, 13.2, 13.3 closes a receiving unit 10.1, 10.2, 10.3, the closure 13.1, 13.2, 13.3 with the window 19.1, 19.2, 19.3 will be located above the receiving unit 10.1, 10.2, 10.3, so that an accommodated gas measuring device 1.1, 1.2, 1.3 is visible through the window 19.1, 19.2, 19.3.

Each closure 13.1, 13.2, 13.3 is rotatably attached to a holder 56.1, 56.2, 56.3. This holder 56.1, 56.2, 56.3 is mechanically connected to the housing 101 and comprises a longitudinal wall. Each locking unit 14.1, 14.2, 14.3 is also mechanically connected to the housing 101 and preferably cannot move relative to the housing 101. The receiving unit 10.1, 10.2, 10.3 is located between the associated locking unit 14.1, 14.2, 14.3 and the holder 56.1, 56.2, 56.3 for the associated closure 13.1, 13.2, 13.3.

Each locking unit 14.1, 14.2, 14.3 is assigned to a closure 13.1, 13.2, 13.3 and can be moved (transferred) to a locking and to a releasing state, independently of any other locking unit. In the locking state, the locking unit 14.1, 14.2, 14.3 locks the associated downward-rotated closure 13.1, 13.2, 13.3. As long as the locking unit 14.1, 14.2, 14.3 is in the locking state, it will not be possible to open the associated closure 13.1, 13.2, 13.3. In this case, a gas measuring device 1.1, 1.2, 1.3 can neither be removed from the receiving unit 10.1, 10.2, 10.3, nor can a gas measuring device be inserted. While the locking unit 14.1, 14.2, 14.3 is in the releasing state, the associated closure 13.1, 13.2, 13.3 can be rotated relative to the housing 101 about the axis of rotation DA.1 and thus moved into the open position.

Each locking unit 14.1, 14.2, 14.3 includes a recess 18.1, 18.2, 18.3. If the associated closure 13.1, 13.2, 13.3 is rotated downward, the projection 17.1, 17.2, 17.3 on this closure 13.1, 13.2, 13.3 will engage in the recess 18.1, 18.2, 18.3. Inside the locking unit 14.1, 14.2, 14.3 there is a movably mounted locking body (not shown). While the locking unit 14.1, 14.2, 14.3 is in the locking state, this locking body engages with the projection 17.1, 17.2, 17.3 and prevents the closure 13.1, 13.2, 13.3 from being rotated upward. The locking body can be pulled out of the projection 17.1, 17.2, 17.3. If the locking unit 14.1, 14.2, 14.3 is in the releasing state, the locking body does not engage with the projection 17.1, 17.2, 17.3.

Each locking unit 14.1, 14.2, 14.3 also contains a controllable actuator, which is likewise not shown, in particular an electric, pneumatic or hydraulic actuator. This actuator can move the locking body relative to the recess 18.1, 18.2, 18.3.

In one embodiment, the actuator is configured to move the locking body back and forth, thereby causing both the locking body to engage with the projection 17.1, 17.2, 17.3 and causing the locking body to be pulled out of the projection 17.1, 17.2, 17.3.

In another embodiment, a mechanical restoring element, for example a spring, is also inserted in each locking unit 14.1, 14.2, 14.3, wherein the restoring element exerts a restoring force. The actuator is configured to move the locking body against the restoring force in the one direction. If the actuator is not active, the restoring force moves the locking body in the opposite direction and holds it in a position.

In one implementation of this other embodiment, the restoring force causes the locking body to be pulled out of the projection 17.1, 17.2, 17.3. This implementation has the following effect in case of a failure: if the actuator fails or is no longer supplied with electrical, hydraulic or pneumatic energy, the restoring force of the mechanical restoring element will transfer the locking unit 14.1, 14.2, 14.3 to the releasing state. In an emergency, the gas measuring device 1.1, 1.2, 1.3 can then be removed from the receiving unit 10.1, 10.2, 10.3.

In the exemplary embodiment, a rod 50.1, 50.2, 50.3 is rotatably inserted in each holder 56.1, 56.2, 56.3. This rod 50.1, 50.2, 50.3 functions as the opening element. Thanks to a recess 57.1, 57.2, 57.3, the rod 50.1, 50.2, 50.3 can rotate relative to the holder 56.1, 56.2, 56.3, namely in a direction perpendicular to the axis of rotation DA.1, cf. FIG. 6. A spring (not shown) is biased to rotate the rod 50.1, 50.2, 50.3 to an upright position and to hold it in this position. The rod 50.1, 50.2, 50.3 effects or supports in particular the steps described below.

As already explained, the locking unit 14.1, 14.2, 14.3, in the locking state, prevents the downward-rotated closure 13.1, 13.2, 13.3 from being opened. The downward-rotated closure 13.1, 13.2, 13.3 holds the rod 50.1, 50.2, 50.3 against the spring force in a pressed-down position. As soon as the locking unit 14.1, 14.2, 14.3 is moved into the releasing state, i.e. the closure 13.1, 13.2, 13.3 is unlocked, the spring rotates the rod 50.1, 50.2, 50.3 upward, i.e. away from the contact element 11.1, 11.2, 11.3. This causes the unlocked closure 13.1, 13.2, 13.3 to open automatically, i.e. to spring open. A user will then know that he/she can now remove the gas measuring device from the receiving unit 10.1, 10.2, 10.3.

Preferably, a rotatably mounted roller 58.1, 58.2, 58.3 is located at the free end of the spring-mounted rod 50.1, 50.2, 50.3. While the closure 13.1, 13.2, 13.3 is rotated upward, the roller 58.1, 58.2, 58.3 on the rod 50.1, 50.2, 50.3 rolls over the downward-facing surface of the closure 13.1, 13.2, 13.3. The roller 58.1, 58.2, 58.3 thus prevents the rod 50.1, 50.2, 50.3 from scratching over the closure 13.1, 13.2, 13.3 or even getting stuck.

In the upright position, the rod 50.1, 50.2, 50.3 prevents the upward-rotated closure 13.1, 13.2, 13.3 from being automatically rotated downward by gravity, i.e. from dropping shut. The closure 13.1, 13.2, 13.3 can be closed against the force of the spring.

A user returns a gas measuring device 1.1, 1.2, 1.3 by inserting the gas measuring device 1.1, 1.2, 1.3 into a suitable receiving unit 10.1, 10.2, 10.3. The following steps are then required: the contact element 11.1, 11.2, 11.3 is rotated downward, and the optional latching mechanism holds the contact element 11.1, 11.2, 11.3 in the downward-rotated position. The closure 13.1, 13.2, 13.3 is rotated downward until the projection 17.1, 17.2, 17.3 engages with the recess 18.1, 18.2, 18.3. The locking unit 14.1, 14.2, 14.3 locks the closure 13.1, 13.2, 13.3.

The following describes an implementation which eliminates the need to rotate the contact element 11.1, 11.2, 11.3 downward first and then the closure 13.1, 13.2, 13.3. Rather, a spacer element 59.1, 59.2, 59.3 is attached to the surface of the closure 13.1, 13.2, 13.3 facing the contact element 11.1, 11.2, 11.3, cf. FIG. 11. Two respective rollers 53.1, 53.2, 53.3 are attached at the free end of the spacer element 59.1, 59.2, 59.3. The spacer element 59.1, 59.2, 59.3 transmits a rotation of the closure 13.1, 13.2, 13.3 towards the receiving unit 10.1, 10.2, 10.3 in such a way onto the contact element 11.1, 11.2, 11.3 that the contact element 11.1, 11.2, 11.3 is also moved towards the receiving unit 10.1, 10.2, 10.3. As the closure 13.1, 13.2, 13.3 is moved towards the contact element 11.1, 11.2, 11.3, the rollers 53.1, 53.2, 53.3 roll over the upward-facing surface of the respective contact element 11.1, 11.2, 11.3.

Preferably, each receiving unit 10.1, 10.2, 10.3 comprises a closure sensor (not shown), which preferably comprises a contact switch. The closure sensor determines whether the associated closure 13.1, 13.2, 13.3 is currently rotated upward or downward. In the exemplary embodiment, the closure 13.1, 13.2, 13.3 can be locked only if it is fully rotated downward.

In the second implementation, a respective mechanical lock 64.1, 64.2, 64.3 is assigned to the locking unit 14.1, 14.2, 14.3. The mechanical lock 64.1, 64.2, 64.3 can be opened with the aid of a matching key (not shown), even if the automatic dispenser 100 is not being supplied with electrical energy or is defective.

The mechanical lock 64.1, 64.2, 64.3 can be moved into a closing state and into an opening state with the aid of the matching key. Preferably, the mechanical lock 64.1, 64.2, 64.3 is embedded in a closure 13.1, 13.2, 13.3 and is connected to the associated locking unit 14.1, 14.2, 14.3 as follows: If the mechanical lock 64.1, 64.2, 64.3 is moved into the opening state with the aid of the key, the associated locking unit 14.1, 14.2, 14.3 is moved into the releasing state, and the closure 13.1, 13.2, 13.3 can be opened or springs open. In one implementation, conversely, the process of moving the locking unit 14.1, 14.2, 14.3 into the locking state also causes the lock 64.1, 64.2, 64.3 to be moved into the closing state.

Instead of a lock, an actuating element may be embedded, wherein the actuating element can be operated from the outside in order to open the closure 13.1, 13.2, 13.3.

In the first implementation (FIG. 4 to FIG. 7), the closing component 25.1, 25.2 comprises the closure 13.1, 13.2 and the locking unit 14.1, 14.2. In the second implementation (FIG. 8 to FIG. 11), the receiving module 26.1, 26.2, 26.3 comprises the receiving unit 10.1, 10.2, 10.3, the closure 13.1, 13.2, 13.3, the locking unit 14.1, 14.2, 14.3, the holder 56.1, 56.2, 56.3 and the lock 64.1, 64.2, 64.3.

The control unit 16.1, 16.2, 16.3, 54, 65 receives and processes the following signals and other information about a gas measuring device 1.1, 1.2, 1.3, which is currently inserted in the receiving unit 10.1, 10.2, 10.3:
In a first implementation, the control unit 16.1, 16.2, 16.3, 54, 65 receives a computer-accessible description via the electrical contact 51.1, 51.2, 51.3, which is stored in a data memory of the inserted gas measuring device 1.1, 1.2, 1.3. This description comprises a unique identifier of the gas measuring device 1.1, 1.2, 1.3 and an item of information about which target gas or target gases this gas measuring device 1.1, 1.2, 1.3 can measure, optionally further information about the gas measuring device 1.1, 1.2, 1.3. In one embodiment, this description also comprises an identification indicating the qualification a user of the gas measuring device 1.1, 1.2, 1.3 must possess in order to be allowed to use the gas measuring device 1.1, 1.2, 1.3. Optionally, this description also comprises a specification of the environmental conditions under which this gas measuring device 1.1, 1.2, 1.3 may be used.
In a second implementation, the control unit 16.1, 16.2, 16.3, 54, 65 determines this item of information by means of a read access to the database 55 for which purpose the Ethernet cable, which is plugged into the port 33, is used.
These two implementations can be combined, in particular to create redundancy.
The control unit 16.1, 16.2, 16.3, 54, 65 receives and processes a signal from the voltage sensor 15.1, 15.2, 15.3 and determines the current charging state of the power supply unit of the inserted gas measuring device 1.1, 1.2, 1.3.
The control unit 16.1, 16.2, 16.3, 54, 65 receives and processes a signal from the sensor of the inserted gas measuring device 1.1, 1.2, 1.3. This signal contains an item of information about the target gas concentration that the sensor of the inserted gas measuring device 1.1, 1.2, 1.3 has measured. The measured target gas concentration refers to the concentration of a target gas in a gas sample, wherein the gas sample originates from a test gas which has been conducted from the container 40 into the measuring chamber of the gas measuring device 1.1, 1.2, 1.3, cf. FIG. 1.
The control unit 16.1, 16.2, 16.3, 54, 65 receives and processes a signal which includes an item of information about the specified and known target gas concentration of the test gas in the container 40.

In the exemplary embodiment, the control unit 16.1, 16.2, 16.3, 54, 65 determines the current status of the inserted gas measuring device 1.1, 1.2, 1.3 as follows:
The control unit 16.1, 16.2, 16.3, 54, 65 determines the current charging state of the power supply unit of the gas measuring device 1.1, 1.2, 1.3. If the determined charging state is less than a specified lower charging state threshold, the gas measuring device 1.1, 1.2, 1.3 cannot currently be used. The power supply unit is usually charged while the gas measuring device 1.1, 1.2, 1.3 is inserted.
Optionally, the control unit 16.1, 16.2, 16.3, 54, 65 predicts a time span. After this time span has elapsed, the power supply unit will be sufficiently charged.
Optionally, the control unit 16.1, 16.2, 16.3, 54, 65 determines how long the gas measuring device 1.1, 1.2, 1.3 can be used at the current charging state, provided the gas measuring device 1.1, 1.2, 1.3 is now removed from the receiving unit 10.1, 10.2, 10.3, and its power supply unit is not charged any further. For this purpose, the control unit 16.1, 16.2, 16.3, 54, 65 uses the determined current charging state as well as a specified and preferably empirically pre-determined consumption of electrical energy per unit of time.
The control unit 16.1, 16.2, 16.3, 54, 65 checks whether the measured target gas concentration deviates from the specified target gas concentration of the test gas in the container 40 by more than a specified tolerance. If the deviation is greater than the tolerance, then the gas measuring device 1.1, 1.2, 1.3 is not measuring correctly, and is therefore not intact.

In one embodiment, the data set for a gas measuring device 1.1, 1.2, 1.3 is supplemented by the determined current state, or the data set is updated.

The control unit 16.1, 16.2, 16.3, 54, 65 is configured to control the display and operating unit 20. Depending on the actuation, information about inserted gas measuring devices 1.1, 1.2, 1.3 is displayed on the screen 21. In particular, an appropriate item of information will be output if an inserted gas measuring device 1.1, 1.2, 1.3 is currently unable to measure a target gas correctly. In particular, the actual target gas concentration of a test gas in the container 40 and the target gas concentration measured by the gas measuring device 1.1, 1.2, 1.3 for a gas sample with this test gas are output. Optionally, if the power supply unit of a gas measuring device 1.1, 1.2, 1.3 has an insufficient charging state, it will be displayed at what time this power supply unit will be sufficiently charged. Optionally, it will be displayed how long the power supply unit of a gas measuring device 1.1, 1.2, 1.3 is currently able to adequately supply this gas measuring device with electrical energy.

The control unit 16.1, 16.2, 16.3, 54, 65 can control each locking unit 14.1, 14.2, 14.3 of the automatic dispenser 100. As a result of the actuation, the locking unit 14.1, 14.2, 14.3 will be in the locking state or in the releasing state. The control unit 16.1, 16.2, 16.3, 54, 65 can also control the respective valve in a contact element 11.1, 11.2, 11.3 and thereby selectively block or release a fluidic connection between a container 40 with a test gas and an inserted gas measuring device 1.1, 1.2, 1.3.

In one embodiment, the process of a gas measuring device 1.1, 1.2, 1.3 being inserted in a receiving unit 10.1, 10.2, 10.3 triggers the following steps: the control unit 16.1, 16.2, 16.3, 54, 65 actuates the valve of the associated contact element 11.1, 11.2, 11.3, the valve opens, and a test gas is conducted from the container 40 into the measuring chamber of the gas measuring device 1.1, 1.2, 1.3 that has just been inserted. The control unit 16.1, 16.2, 16.3, 54, 65 compares the target gas concentration measured by the inserted gas measuring device 1.1, 1.2, 1.3 with the actual target gas concentration.

A carrier 67 is mounted on a side wall of the housing 101 such that the carrier 67 is rotatable relative to the housing 101 about an axis of rotation DA.3 between two end positions, cf. FIG. 8 to FIG. 10. The carrier 67 locks into place in each end position. An RFID reader device 60 and a scanner 66, for example a barcode scanner or QR code scanner, are embedded in the carrier 67. Because the carrier 67 is rotatable, it is made easier for a user to rotate the carrier 67 into a desired position. A sleeve 69 is also arranged on the carrier 67, cf. FIG. 10. A scanner cable (not shown) is guided through this sleeve 69 and during operation is plugged into the socket 35 in the housing 101.

In one embodiment, the RFID reader device 60 and the scanner 66 have the following two tasks:
On the one hand, a user who wants to remove a gas measuring device 1.x (x=1, 2, . . . ), or a maintenance technician, can authorize himself/herself either with the RFID reader device 60 or with the scanner 66.
On the other hand, a user who wants to return a gas measuring device 1.y, or a maintenance technician, can cause the RFID reader device 60 or the scanner 66 to read a machine-readable identifier on a surface of this gas measuring device 1.y.

At the beginning of an operation of the automatic dispenser 100, the following situation is preferably established: each closure 13.1, 13.2, 13.3 and each contact element 11.1, 11.2, 11.3 are rotated downward. Each closure 13.1, 13.2, 13.3 is locked. At least once, a sequence consisting of the following steps is performed:

A person wants to use a gas measuring device. This person will hereinafter be referred to as “the user.” The user authorizes himself/herself at the display and operating unit 20 and thereby logs in. For example, the user enters a username and password. Or an identifier on an employee ID card or a biometric feature is determined. The following steps will be performed only if the user has successfully authorized himself/herself.

Optionally, the user can specify by means of the display and operating unit 20 the target gas for which he/she needs a gas measuring device. Optionally, the user specifies the maximum duration for which he/she wants to use the gas measuring device. Optionally, the user specifies an identification for an environmental condition for an intended use of the gas measuring device, for example the highest expected ambient temperature or ambient humidity.

In one embodiment, a qualification profile is stored in a data memory for each set of possible users. This qualification profile specifies which gas measuring devices this user is allowed to use.

The event in which a user has successfully authorized himself/herself and requests the dispensing of a gas measuring device triggers the following step: the control unit 16.1, 16.2, 16.3, 54, 65 searches for a gas measuring device that currently cumulatively exhibits the following properties:
The gas measuring device is inserted in a receiving unit 10.1, 10.2, 10.3.
The gas measuring device is configured to measure the concentration of the target gas for which the user needs a gas measuring device.
The gas measuring device is intact and can therefore measure the concentration of the target gas with sufficient accuracy. This means that the gas measuring device has measured the concentration of this target gas in a gas sample of the test gas from the container 40, and the measured target gas concentration deviates from the actual target gas concentration by no more than the specified absolute or percentage tolerance.
The power supply unit of the gas measuring device has a sufficiently high charging state, i.e. a charging state above the specified lower charging state threshold.
In one embodiment, the user has specified a maximum duration for use of the gas measuring device, and the charging state of the gas measuring device is sufficient for at least this maximum duration.
In one embodiment, the user has specified at least one expected environmental condition, and the gas measuring device is suitable for this environmental condition, i.e. it can measure correctly in particular at the specified ambient temperature or ambient humidity.
In one embodiment, a qualification profile is stored for the user who has authorized himself/herself at the display and operating unit 20. According to this qualification profile, the user is qualified and suitable to use the gas measuring device.

If a gas measuring device cumulatively meets these conditions, then it is usable.

If the control unit 16.1, 16.2, 16.3, 54, 65 does not find a usable gas measuring device, the control unit 16.1, 16.2, 16.3, 54, 65 causes a corresponding error message to be displayed on the screen 21. If the control unit 16.1, 16.2, 16.3, 54, 65 finds several usable gas measuring devices, the control unit 16.1, 16.2, 16.3, 54, 65 automatically selects one, for example the one with the currently highest charging state or with the best concentration measurement (the one with the lowest error).

The following describes the steps that are carried out after the control unit 16.1, 16.2, 16.3, 54, 65 has found a usable gas measuring device 1.x (x=1, 2, . . . ) and, if several usable gas measuring devices are found, has selected one of them. The gas measuring device 1.x is inserted in the receiving unit 10.x. Initially, the closure 13.x closes the receiving unit 10.x, and the locking unit 14.x locks the closure 13.x.

The control unit 16.1, 16.2, 16.3, 54, 65 causes the following steps to be carried out:
The locking unit 14.x is moved into the releasing state.
Preferably, the closure 13.x opens automatically, for example springs open. Alternatively, a message is displayed on the screen 21 indicating that the closure 13.x is no longer locked, but is released. This message comprises a visually perceptible identification of the closure 13.x or of the receiving unit 10.x.
The user can now rotate the contact element 11.x upward and remove the gas measuring device 1.x.
It is checked whether the gas measuring device 1.x has been removed from the receiving unit 10.x. Once this is the case, the data set for the gas measuring device 1.x will be updated accordingly.

The embodiment just described ensures that the user can remove only a usable gas measuring device. An embodiment allows a maintenance technician to remove a gas measuring device that is not necessarily intact. A maintenance technician is a person who is authorized and able to remove, inspect, and, if necessary, repair or replace a gas measuring device.

The maintenance technician authorizes himself/herself at the automatic dispenser 100. In a first embodiment, the step of a maintenance technician authorizing himself/herself triggers the following step: the control unit 16.1, 16.2, 16.3, 54, 65, by appropriate actuation, causes each locking unit 14.1, 14.2, 14.3 to be moved into the releasing state. This allows every closure 13.1, 13.2, 13.3 to be opened. The maintenance technician can remove each inserted gas measuring device 1.1, 1.2, 1.3.

In a second embodiment, if a maintenance technician has authorized himself/herself, the control unit 16.1, 16.2, 16.3, 54, 65 then determines each inserted gas measuring device that has at least one of the following properties:
The gas measuring device is not able to measure a target gas concentration correctly. In particular, the gas measuring device has measured the concentration of a target gas in a gas sample, wherein this gas sample originates from the test gas in the container 40, and the measured target gas concentration deviates from the actual target gas concentration in the test gas by more than the specified tolerance.
The control unit 16.1, 16.2, 16.3, 54, 65 has found that the gas measuring device has another fault, for example, its power supply unit, its output unit, or its alarm unit are defective.

According to the second embodiment, the control unit 16.1, 16.2, 16.3, 54, 65 effects the following: the respective closure of each receiving unit in which a defective gas measuring device is inserted is unlocked and thereby released. The maintenance technician can remove the or each defective gas measuring device.

In a third embodiment, the maintenance technician selects a receiving unit 10.z (z=1, 2, . . . ), for example by a corresponding user input on the screen 21. The locking unit 14.z is moved into the releasing state. The closure 13.z can be opened or springs open. The maintenance technician can remove the inserted gas measuring device 1.z from the receiving unit 10.z.

After use, a user returns a gas measuring device 1.y by inserting it into a receiving unit 10.y (y=1, 2, . . . ) of the automatic dispenser 100. To insert the gas measuring device 1.y, the user rotates the associated closure 13.y upward, or the closure 13.y springs open by itself. In addition, the user rotates the contact element 11.y upward.

After insertion, the user closes the associated closure 13.y. The step of closing the closure 13.y causes the contact element 11.y to be closed as well due to the mechanical coupling described above. Once the closure 13.y is closed, the control unit 16.1, 16.2, 16.3, 54, 65 or a restoring element causes the associated locking unit 14.y to lock the closure 13.y.

In one embodiment, if no gas measuring device is inserted in a receiving unit 10.y, the associated closure 13.y is unlocked. In a different embodiment, however, the locking unit 14.y first locks the closure 13.y for the free receiving unit 10.y.

According to one implementation of the alternative embodiment, the user first brings the gas measuring device 1.y, which he/she wishes to return, into the vicinity of the RFID reader device 60 or the scanner 66. The RFID reader device 60 or the scanner 66 reads a machine-readable identifier on a surface of the gas measuring device 1.y. If this identifier is not found or cannot be read, the control unit 65 causes a corresponding error message to be issued.

In another implementation, the automatic dispenser 100 and each gas measuring device each comprise a communication unit for wireless data communication over a relatively short distance, for example via Bluetooth Low Energy (BLE) or, generally, Near-Field Communication (NFC). The communication unit of the automatic dispenser 100 receives a message from a gas measuring device that has been brought close to the automatic dispenser 100. This message comprises a unique identifier of the gas measuring device.

As soon as a gas measuring device 1.y to be returned has been detected, the control unit 65 automatically searches for a suitable receiving unit 10.y. It looks in particular for a free receiving unit 10.y with suitable dimensions and a suitable electrical coupling point. If no suitable receiving unit is found, the control unit 65 causes a corresponding error message to be issued. If a suitable receiving unit 10.y is found, the control unit 65 causes the associated locking unit 14.y to be moved into the releasing state. The associated closure 13.y can be opened or springs open by itself.

The automatic dispenser 100 is connected to the container 40 at least temporarily, cf. FIG. 1. In one embodiment, the step of inserting the gas measuring device 1.y into the receiving unit 10.y, rotating the contact element 11.y and the closure 13.y towards the receiving unit 10.y, and moving the locking unit 14.y to the locking state, triggers the following step: a test gas from the container 40 is conducted to the contact element 11.y and further to the measuring chamber of the inserted gas measuring device 1.y. The gas measuring device 1.y measures the target gas concentration in the test gas, and the measured target gas concentration is compared with the actual target gas concentration. This ensures that the gas measuring device 1.y is checked immediately after insertion. Preferably, for this check, the valve mentioned above in the contact element 11.y is opened, thereby releasing the gas supply element 12.y.

Further above, with reference to FIG. 2, a database 55 was mentioned in which a data set is stored for each gas measuring device. In one embodiment, the process just described, in which the RFID reader device 60 or the scanner 66 has read the identifier of a gas measuring device that a user wants to return, triggers the following steps: the data set for this gas measuring device 1.y in the database 55 is updated accordingly. The data set is at least supplemented with the information that the gas measuring device 1.y is now located in the automatic dispenser 100, preferably additionally with the information at what time the gas measuring device 1.y was inserted into the receiving unit 10.y, and/or with the information into which receiving unit it was inserted.

Often, a user will take a gas measuring device from the automatic dispenser 100 to use it in an environment that may be dangerous to humans. In many cases, the gas measuring device, in particular a housing of the gas measuring device, absorbs at least one fluid, for example a long-chain hydrocarbon. As a consequence of this often unavoidable event, gas escapes from the gas measuring device 1.y if the gas measuring device 1.y is reinserted in a receiving unit of the automatic dispenser 100. In one implementation, a sensor permanently assigned to the receiving unit can detect this gas. The control unit 16.1, 16.2, 16.3, 54, 65 causes a corresponding message to be generated and issued.

In one embodiment, the step of inserting a gas measuring device 1.y into a receiving unit 10.y triggers the following steps: the gas measuring device is automatically disinfected and/or cleaned, for example by means of a purging gas or by means of disinfecting electromagnetic radiation, in particular ultraviolet radiation. This embodiment reduces the risk of an inserted contaminated gas measuring device 1.y contaminating other gas measuring devices in the automatic dispenser 100. While the gas measuring device 1.y in the receiving unit 10.y is disinfected and/or cleaned, the locking unit 14.y preferably locks the closure 13.y.

The following embodiment takes into account the possibility that a returned gas measuring device 1.y is mechanically damaged. The most common types of mechanical damage are the following:
The screen is broken or scratched.
A holder with which the gas measuring device can be attached to a user's protective clothing is damaged.
A crack has appeared in the casing.
The gas measuring device is dirty, in particular smeared.

In one embodiment, the user who wants to return the gas measuring device is asked to indicate whether the gas measuring device is damaged or not. The automatic dispenser 100 captures a corresponding user input. The following alternative embodiment eliminates the need for the user to perform such a check and input.

According to this alternative embodiment, the automatic dispenser 100 comprises a camera and a signal-processing image evaluation unit. For example, the scanner 66 is implemented as a camera or comprises a camera. The image evaluation unit can be part of the control unit 65. In one embodiment, this camera can read and decode a barcode or QR code, or even an alphanumeric identifier. The barcode or QR code or the alphanumeric identifier uniquely identifies the gas measuring device 1.y. It is also possible for an NFC reader device, in particular the RFID reader device 60, to read a corresponding chip in the gas measuring device 1.y and thereby determine the identifier of the gas measuring device 1.y. In both embodiments, the camera is used to automatically detect mechanical damage to the gas measuring device 1.y.

The camera 66 produces images of the gas measuring device 1.y to be returned, which is located outside the automatic dispenser 100 and sufficiently close to the camera. For example, the automatic dispenser 100 comprises a plate or tray or another receptacle. The RFID reader device 60 can itself be integrated into such a plate. If the gas measuring device 1.y to be returned is placed on this support, it will be correctly positioned relative to the camera 66. The camera 66 produces at least one image of the gas measuring device 1.y. Preferably, the camera 66 produces an image of the front side and an image of the back side of the gas measuring device 1.y, wherein the user positions the gas measuring device 1.y such that the front side or the back side faces the camera 66.

The image evaluation unit evaluates this image and automatically decides whether mechanical damage to the gas measuring device 1.y can be detected in at least one image. If so, a corresponding message is issued, and/or the data set in the database 55 for this gas measuring device 1.y is updated accordingly. The image evaluation unit preferably comprises a trained classifier. This classifier was previously trained using a machine learning method. The classifier is particularly preferably trained using an annotated sample. This sample comprises a plurality of images, wherein each image shows a gas measuring device and wherein an annotation is associated with the image. The annotation describes whether the gas measuring device shown in the image has a mechanical damage or not, and if so, what kind of mechanical damage is present.

In one embodiment, at least one receiving unit is assigned a weighing device. The step of a gas measuring device 1.y having been inserted into this receiving unit triggers the following step: the weighing device measures the weight of the inserted gas measuring device 1.y. The measured weight is compared with a given target weight, wherein this target weight is, for example, part of the data set for this gas measuring device 1.y in the database 55. If the measured weight deviates significantly from the given target weight, this is an indication of an error. For example, the returned gas measuring device 1.y is missing a sufficiently heavy part, in particular a battery.

List of reference signs

| | |
|---|---|
| 1.1, 1.2, 1.3 | Gas measuring device, located in the receiving unit 10.1, |
| | 10.2, 10.3, comprises the gas inlet 2.1, 2.2, 2.3, the display |
| | unit 3.1, 3.2, 3.3 and the operating elements 4.1, 4.2, 4.3 as |
| | well as a measuring chamber, a sensor and its own power |
| | supply unit |
| 2.1, 2.2, 2.3 | Gas inlet of the gas measuring device 1.1, 1.2, 1.3 |
| 3.1, 3.2, 3.3 | Display unit of the gas measuring device 1.1, 1.2, 1.3 |
| 4.1, 4.2, 4.3 | Operating elements of the gas measuring device 1.1, 1.2, |
| | 1.3 |
| 10.1, 10.2, | Receiving unit, is configured to accommodate a gas |
| 10.3 | measuring device 1.1, 1.2, 1.3, can be closed by a closure |
| | 13.1, 13.2, 13.3, comprises the contact element 11.1, 11.2, |
| | 11.3 and the electrical contact 51.1, 51.2, 51.3 |
| 11.1, 11.2, | Contact element of the receiving unit 10.1, 10.2, 10.3, |
| 11.3 | receives the gas supply element 12.1, 12.2, 12.3, rotatable |
| | relative to the housing 101 about the axis of rotation DA.2 |
| 12.1, 12.2, | Gas supply element in the contact element 11.1, 11.2, 11.3, |
| 12.3 | connected to the gas line 30, belongs to the fluid guidance |
| | arrangement |
| 13.1, 13.2, | Closure for the receiving unit 10.1, 10.2, 10.3, rotatable |
| 13.3 | relative to the housing 101 about the axis of rotation DA.1, |
| | comprises the projection 17.1, 17.2, 17.3, the window 19.1, |
| | 19.2, 19.3 and the spacer element 59.1, 59.2, 59.3 |
| 14.1, 14.2, | Locking unit for the closure 13.1, 13.2, 13.3 comprises the |
| 14.3 | recess 18.1, 18.2, 18.3 as well as a locking body, an |
| | actuating element and optionally a restoring element, can be |
| | moved into a locking and a releasing state, according to the |
| | second implementation it comprises the mechanical lock |
| | 64.1, 64.2, 64.3 |
| 15.1, 15.2, | Voltage sensor of the receiving unit 10.1, 10.2, 10.3, |
| 15.3 | measures the charging state of the power supply unit of an |
| | inserted gas measuring device 1.1, 1.2, 1.3 |
| 16.1, 16.2, | Evaluation unit of the receiving unit 10.1, 10.2, 10.3, |
| 16.3 | compares a measured target gas concentration with an |
| | actual target gas concentration |
| 17.1, 17.2, | Projection on the closure 13.1, 13.2, 13.3, engages with the |
| 17.3 | recess 18.1, 18.2, 18.3 if the closure 13.1, 13.2, 13.3 is |
| | in the closing position |
| 18.1, 18.2, | Recess in the locking unit 14.1, 14.2, 14.3, receives the |
| 18.3 | projection 17.1, 17.2, 17.3 if the closure 13.1, 13.2, |
| | 13.3 is in the closing position |
| 19.1, 19.2, | Window in the closure 13.1, 13.2, 13.3, through which an |
| 19.3 | inserted gas measuring device 1.1, 1.2, 1.3 is visible |
| 20 | Display and operating unit, comprises the screen 21; in one |
| | embodiment the screen 70 and the power button 22, |
| | functions as the input unit |
| 21 | Touchscreen of the display and operating unit 20 |
| 22 | Power button of the display and operating unit 20 |
| 25.1, 25.2 | Closing component according to the first implementation of |
| | the present disclosure, surrounds the receiving unit 10.1, |
| | 10.2, comprises the closure 13.1, 13.2, the locking |
| | unit 14.1, 14.2 and the holder 56.1, 56.2 |
| 26.1, 26.2, | Receiving module according to the second implementation, |
| 26.3 | accommodates the receiving unit 10.1, 10.2, 10.3, |
| | comprises the closure 13.1, 13.2, 13.3, the locking |
| | unit 14.1, 14.2, 14.3 and the holder 56.1, 56.2, 56.3 |
| 30 | Gas line, connects the housing gas inlet 31 with the gas |
| | supply elements 12.1, 12.2, 12.3, belongs to the fluid |
| | guidance arrangement |
| 31 | Housing gas inlet in the housing 101, start of the gas line 30 |
| 32 | Additional housing gas inlet in the housing 101 |
| 33 | Port in the housing 101 for an Ethernet cable, the cable |
| | connecting the housing 101 to the database 55 |
| 34 | Socket in the housing 101 for a line connecting the |
| | automatic dispenser 100 to a stationary power supply |
| | network |
| 35 | Socket in the housing 101 for a scanner cable |
| 40 | Container in the form of a bottle containing test gas, |
| | connected to the line 41 |
| 41 | Fluid guidance unit in the form of a line connecting the |
| | container 40 to the housing gas inlet 31 |
| 50.1, 50.2, | Spring-loaded rod, opens the closure 13.1, 13.2, 13.3, |
| 50.3 | transmits a closing of the closure 13.1, 13.2, 13.3 to the |
| | contact element 11.1, 11.2, 11.3, carries the roller 58.1, |
| | 58.2, 58.3 at its free end, functions as an opening element |
| 51.1, 51.2, | Electrical contact in the receiving unit 10.1, 10.2, 10.3, |
| 51.3 | electrically contacts an inserted gas measuring device 1.1, |
| | 1.2, 1.3 |
| 53.1, 53.2, | Roller, rotatably mounted at the free end of the spacer |
| 53.3 | element 59.1, 59.2, 59.3 |
| 54 | Control device of the automatic dispenser 100 |
| 55 | Database containing data sets for the gas measuring |
| | devices, connected to test station 80 via an Ethernet cable |
| 56.1, 56.2, | Holder to which the closure 13.1, 13.2, 13.3 is rotatably |
| 56.3 | attached, is mechanically connected to the housing 101, |
| | holds the rod 50.1, 50.2, 50.3, belongs to the closing |
| | component 25.1, 25.2 and to the receiving module 26.1, |
| | 26.2, 26.3 |
| 57.1, 57.2, | Recess in the holder 56.1, 56.2, 56.3 for the rod 50.1, 50.2, |
| 57.3 | 50.3 |
| 58.1, 58.2, | Roller, rotatably mounted at the free end of the rod 50.1, |
| 58.3 | 50.2, 50.3 |
| 59.1, 59.2, | Spacer element, attached to the closure 13.1, 13.2, 13.3, |
| 59.3 | comprises the roller 53.1, 53.2, 53.3, rotates the contact |
| | element 11.1, 11.2, 11.3 downward |
| 60 | RFID reader device, attached to the carrier 67 |
| 61 | Protective flap for the display and operating unit 20, |
| | accommodates the screen 70, comprises the window 62, is |
| | rotatable relative to the housing 101 about the axis of |
| | rotation DA.1 |
| 62 | Window of the protective flap 61 through which the screen |
| | 21 is visible |
| 63 | Mechanical lock for the protective flap 61 |
| 64.1, 64.2, | Mechanical lock for the closure 13.1, 13.2, 13.3 |
| 64.3 | |
| 65 | Control unit, performs the functions of the control |
| | device 54 and the evaluation units 16.1, 16.2, 16.3 |
| 66 | Scanner, for example barcode scanner, attached to the |
| | carrier 67, can comprise a camera |
| 67 | Carrier for the RFID reader device 60 and the scanner 66, |
| | arranged laterally on the housing 101, rotatably mounted |
| | relative to the housing 101 and the axis of rotation DA.3 |
| 69 | Sleeve on the carrier 67 through which a scanner cable is |
| | guided |
| 70 | Touchscreen, embedded in the protective flap 61 |
| 71 | Hinge by which the protective flap 61 is attached to the |
| | housing 101 |
| 80 | Test station, comprising the display and operating unit 20, |
| | the receiving units 10.1, 10.2, 10.3, the gas line 30, the |
| | housing gas inlets 31, 32 and the housing 101 |
| 100 | Automatic dispenser, comprises the test station 80, the |
| | closures 13.1, 13.2, 13.3, the locking units 14.1, 14.2, 14.3, |
| | the voltage sensors 15.1, 15.2, 15.3, and, according to the |
| | first implementation, the evaluation units 16.1, 16.2, 16.3 |
| | and the control device 54, and, according to the second |
| | implementation, the control unit 65 |
| 101 | Housing of the test station 80, accommodates the housing |
| | gas inlets 31 and 32, the port 33, and the sockets 34 and 35 |
| DA.1 | Axis of rotation about which the closure 13.1, 13.2, 13.3 is |
| | rotatable relative to the housing 101, parallel to the axes of |
| | rotation DA.2 and DA.3 |
| DA.2 | Axis of rotation about which the contact element 11.1, 11.2, |
| | 11.3 is rotatable relative to the housing 101 |
| DA.3 | Axis of rotation about which the carrier 67 is rotatably |
| | mounted relative to the housing 101 |

1. An automatic dispenser for a gas measuring device, wherein the automatic dispenser comprises:
a receiving unit;
a closure that is movable relative to the receiving unit back and forth between a closing position and an open position;
a locking unit that can be moved into a locking state and into a releasing state;
a housing gas inlet;
a fluid guidance arrangement;
an input unit; and
a signal-processing control unit,
wherein a gas measuring device can be inserted into and removed from the receiving unit,
wherein the fluid guidance arrangement connects the housing gas inlet to the receiving unit such that:
while a gas measuring device is inserted in the receiving unit, the fluid guidance arrangement establishes a fluidic connection between the housing gas inlet and the inserted gas measuring device,
wherein the closure in the closing position prevents the gas measuring device from being removed from the receiving unit and, in the open position, enables removal of the gas measuring device from the receiving unit,
wherein the locking unit in the locking state prevents the closure, in the closing position, from being moved into the open position, and wherein the locking unit in the releasing state enables a movement of the closure from the closing position into the open position,
wherein the input unit is configured to capture a user input comprising information that dispensing of a gas measuring device is desired,
wherein the control unit is configured to check, in response to capturing a user input, whether a usable gas measuring device is inserted in the receiving unit,
wherein a given gas measuring device is only usable if the given gas measuring device is intact, and wherein the given gas measuring device is intact if the given gas measuring device can correctly measure a concentration of a given target gas up to a specified tolerance, and
wherein the control unit, in response to detecting that a usable gas measuring device is inserted in the receiving unit, is further configured to cause the locking unit for the closure to be moved into the releasing state.

2. The automatic dispenser of claim 1, wherein:
the receiving unit comprises an opening element,
wherein the opening element is configured to move the closure from the closing position into the open position.

3. The automatic dispenser of claim 1, wherein:
the automatic dispenser comprises a voltage sensor and the receiving unit comprises an electrical contact,
wherein, while a gas measuring device is inserted in the receiving unit,
an electrical connection between a power supply unit of the inserted gas measuring device and the voltage sensor is at least temporarily established or can be established by means of the electrical contact,
wherein the voltage sensor is configured to measure a current charging state of the power supply unit of the inserted gas measuring device, and
wherein the control unit is configured to determine that the inserted gas measuring device is currently not usable if the measured current charging state is less than a given charging state threshold.

4. The automatic dispenser of claim 1, wherein:
the captured user input additionally comprises an item of information about a desired property of a gas measuring device, and
the control unit is configured to:
check whether a gas measuring device that has the desired property is inserted in the receiving unit; and
classify the inserted gas measuring device as usable only if the gas measuring device is intact and has the desired property.

5. The automatic dispenser of claim 1, wherein:
the receiving unit comprises a contact element that is movable back and forth between a contacting position and a spaced-apart position relative to a gas measuring device inserted in the receiving unit,
wherein, while the contact element is in the contacting position, the fluid guidance arrangement establishes a fluidic connection between the housing gas inlet and the inserted gas measuring device,
wherein, while the contact element is in the spaced-apart position, the fluidic connection between the housing gas inlet and the inserted gas measuring device is interrupted, and
wherein the contact element is mechanically coupled to the closure such that a movement of the closure from the open position into the closing position causes the contact element to be moved from the spaced-apart position into the contacting position.

6. The automatic dispenser of claim 1, wherein:
the automatic dispenser comprises a manually operable actuating element,
wherein the actuating element is connected to the locking unit such that an actuation of the actuating element causes the locking unit to be moved into the releasing state.

7. The automatic dispenser of claim 1, wherein:
the automatic dispenser comprises a camera and a signal-processing image evaluation unit,
wherein the camera is configured to produce an image of a given gas measuring device,
wherein the camera is positioned such that the image shows the given gas measuring device while the given gas measuring device is located outside of the automatic dispenser, and
wherein the image evaluation unit is configured to automatically determine, by evaluating the image of the given gas measuring device, whether the given gas measuring device has any visually detectable damage.

8. The automatic dispenser of claim 1, wherein:
the automatic dispenser is configured to cause a gas sample to flow from the housing gas inlet through the fluidic connection to a gas measuring device that is inserted in the receiving unit, the gas sample comprising the target gas with a given target gas concentration, and
the control unit is configured to:
cause the inserted gas measuring device to measure the target gas concentration in the gas sample;
compare the measured target gas concentration with the given target gas concentration; and
determine that the gas measuring device is not intact if the measured target gas concentration deviates from the given target gas concentration by more than a specified tolerance.

9. The automatic dispenser of claim 8, wherein:
the control unit is configured to cause, in response to a triggering event:
the measurement of the target gas concentration;
the comparison of the measured target gas concentration with the given target gas concentration; and
the determining that the gas measuring device is not intact,
wherein the input unit is configured to capture a further user input, wherein the further user input comprises information that a check of the inserted gas measuring device is desired, and the triggering event is at least one of:
the capturing of the further user input; and
an event in which, since a most recent time an inserted gas measuring device measured the target gas concentration, a specified upper time duration threshold has elapsed.

10. A system comprising:
an automatic dispenser;
a gas measuring device;
an external fluid guidance unit; and
a container comprising a test gas,
wherein the automatic dispenser comprises:
a receiving unit;
a closure that is movable relative to the receiving unit back and forth between a closing position and an open position;
a locking unit that can be moved into a locking state and into a releasing state;
a housing gas inlet;
a fluid guidance arrangement;
an input unit; and
a signal-processing control unit,
wherein the gas measuring device is configured to be inserted into and be removed from the receiving unit,
wherein the fluid guidance arrangement connects the housing gas inlet to the receiving unit such that:
while a gas measuring device is inserted in the receiving unit, the fluid guidance arrangement establishes a fluidic connection between the housing gas inlet and the inserted gas measuring device,
wherein the closure in the closing position prevents the gas measuring device from being removed from the receiving unit and, in the open position, enables removal of the gas measuring device from the receiving unit,
wherein the locking unit in the locking state prevents the closure, in the closing position, from being moved into the open position, and wherein the locking unit in the releasing state enables a movement of the closure from the closing position into the open position,
wherein the input unit is configured to capture a user input comprising information that dispensing of a gas measuring device is desired,
wherein the control unit is configured to check, in response to capturing a user input, whether the gas measuring device inserted in the receiving unit is usable,
wherein a given gas measuring device is only usable if the given gas measuring device is intact, and wherein the given gas measuring device is intact if the given gas measuring device can correctly measure a concentration of a given target gas up to a specified tolerance, and
wherein the control unit, based on detecting that the gas measuring device inserted in the receiving unit is usable, is further configured to cause the locking unit for the closure to be moved into the releasing state,
wherein the test gas comprises a target gas with a given target gas concentration,
wherein the external fluid guidance unit is configured to establish a fluidic connection between the container and the housing gas inlet,
wherein, while the gas measuring device is inserted in the receiving unit:
the system is configured to cause a gas sample comprising the test gas to flow from the container to the inserted gas measuring device, and
wherein the control unit is configured to:
cause the inserted gas measuring device to measure the target gas concentration in the gas sample;
compare the measured target gas concentration with the given target gas concentration; and
determine that the gas measuring device is not intact if the measured target gas concentration deviates from the given target gas concentration by more than a specified tolerance.

11. A dispensing method for dispensing a gas measuring device:
wherein the dispensing method is carried out using an automatic dispenser,
wherein the automatic dispenser comprises:
a receiving unit configured to accommodate the gas measuring device;
a closure;
a locking unit;
a housing gas inlet;
a fluid guidance arrangement;
an input unit; and
a signal-processing control unit,
wherein the fluid guidance arrangement connects the housing gas inlet to the receiving unit such that while a gas measuring device is inserted in the receiving unit, the fluid guidance arrangement establishes a fluidic connection between the housing gas inlet and the inserted gas measuring device,
wherein, prior to carrying out the dispensing method:
the closure is in a closing position relative to the receiving unit and prevents the inserted gas measuring device from being removed from the receiving unit; and
the locking unit is in a locking state and prevents the closure from being moved into an open position,
wherein the dispensing method comprises the steps of:
with the input unit, capturing a user input comprising information that a dispensing of a gas measuring device is desired,
in response to capturing the user input, with the control unit, checking whether a usable gas measuring device is inserted in the receiving unit,
wherein a given gas measuring device is usable only if the given gas measuring device is intact, and wherein the given gas measuring device is intact if the given gas measuring device is able to correctly measure a target gas concentration up to a prespecified tolerance,
in response to a determination that the gas measuring device inserted in the receiving unit is usable, with the control unit, causing the locking unit to be moved into a releasing state,
wherein, upon movement of the locking unit into the releasing state, the closure is released.

12. The dispensing method of claim 11, further comprising:
prior to performing the dispensing method:
inserting the gas measuring device into the receiving unit;
establishing, by an external fluid guidance unit, a fluidic connection between a container comprising a test gas and the housing gas inlet,
wherein the test gas comprises a target gas with a given target gas concentration; and
causing a gas sample comprising the test gas to flow through the external fluid guidance unit and through the fluid guidance arrangement to the inserted gas measuring device,
wherein the method further comprises subsequently:
with the inserted gas measuring device, measuring the target gas concentration in the gas sample, and
with the control unit, checking whether the inserted gas measuring device is intact,
wherein the control unit determines that the inserted gas measuring device is intact if the measured target gas concentration deviates from the given target gas concentration by no more than the prespecified tolerance.

13. A return method for returning a gas measuring device to an automatic dispenser:
wherein the automatic dispenser comprises:
a receiving unit configured to accommodate the gas measuring device;
a closure;
a locking unit;
a reader device; and
a signal-processing control unit,
wherein, prior to carrying out the return method:
no gas measuring device is inserted in the receiving unit;
the closure is in a closing position relative to the receiving unit and prevents the gas measuring device from being inserted into the receiving unit; and
the locking unit is in a locking state and prevents the closure from being moved into an open position,
wherein the return method includes the steps of
with the reader device, capturing a machine-readable identifier of the gas measuring device that is to be returned,
in response to capturing the machine-readable identifier, with the control unit, checking whether the receiving unit is able to accommodate the gas measuring device, and
in response to a determination that the receiving unit is able to accommodate the gas measuring device, with the control unit, causing the locking unit to be moved into a releasing state,
wherein, upon movement of the locking unit into the releasing state, the closure is released.

14. The return method of claim 13, wherein:
the automatic dispenser additionally comprises:
a housing gas inlet; and
a fluid guidance arrangement,
wherein the fluid guidance arrangement establishes a fluidic connection between the housing gas inlet and the receiving unit, and
wherein, based on the gas measuring device having been inserted into the receiving unit, the steps are triggered that:
a fluidic connection is established between a container comprising a test gas and the housing gas inlet by an external fluid guidance unit,
wherein the test gas comprises a target gas with a given target gas concentration;
a gas sample comprising the test gas flows through the external fluid guidance unit and through the fluid guidance arrangement to the inserted gas measuring device;
with the inserted gas measuring device, measuring the target gas concentration in the gas sample; and
with the control unit, checking whether the inserted gas measuring device is intact,
wherein the control unit determines that the gas measuring device is intact if the measured target gas concentration deviates from the given target gas concentration by no more than a specified tolerance.

15. The return method of claim 13, wherein:
the automatic dispenser comprises a camera and a signal-processing image evaluation unit,
wherein the camera is positioned such that an image taken by the camera shows the gas measuring device while the gas measuring device is located outside the automatic dispenser, and
wherein the step of the reader device capturing the machine-readable identifier of the gas measuring device to be returned additionally triggers the steps of
with the camera generating an image of the gas measuring device, and
with the image evaluation unit, automatically determining, by evaluating the image of the gas measuring device, whether the gas measuring device has any visually detectable damage.
